(19) United States
(12) Patent Application Publication
(10) Pub. No.: US 2004/0091985 A1
(43) Pub. Date: May 13, 2004

(54) NAD phosphite oxidoreductase, a novel catalyst from bacteria for regeneration of NAD(P)H

(76) Inventors: William W. Metcalf, Savoy, IL (US); Wilfred A. van der Donk, Champaign, IL (US); Jennifer M. Vrtis, Urbana, IL (US); Andrea K. White, Urbana, IL (US); Amaya M. Garcia Costas, Billings, MT (US)

Correspondence Address: BARNES & THORNBURG, P.O. Box 2786, Chicago, IL, 60690-2786, US

(21) Appl. No.: 10/371,701

(22) Filed: Feb. 21, 2003

Related U.S. Application Data
(60) Provisional application No. 60/359,091, filed on Feb. 22, 2002.

(51) Int. Cl. C12N 9/02; C12P 19/30; C07H 21/04; C12P 21/02; C12N 5/06

(52) U.S. Cl. 435/189; 435/69.1; 435/320.1; 435/325; 435/89; 536/23.2

(57) ABSTRACT

A gene encoding an enzyme required for operation of a novel biochemical pathway for oxidation of the reduced phosphorus (P) compound phosphite was cloned from Pseudomonas and also found in other bacteria. The enzyme (designated PtxD) was overproduced in the host Escherichia coli by use of a recombinant system and purified to homogeneity via a two-step affinity protocol and characterized. The enzyme stoichiometrically produces NADH and phosphate from NAD and phosphite. Mechanistic studies indicate stereoselective transfer of hydride from phosphite to the Re-face of NAD⁺ with observed steady-state kinetic isotope effects of 2.1 on Vₘₐₓ and 1.8 on Vₘₐₓ/Km. The novel enzyme is useful for methods requiring regenerating the cofactor NADH, for use in synthetic oxidoreductases, and to synthesize chiral compounds, complex carbohydrates, and isotopically-labelled compounds.

The application claims priority from U.S. Ser. No. 60/359,091 filed Feb. 22, 2002, the discosure of which is incorporated herein by reference.

The U.S. Government may have rights in this invention due to partial support of NIH GM 59334, NIH GM51334, NRSA 5 F32 GM 16504-3.

### BACKGROUND

A gene encoding an enzyme required for operation of a novel biochemical pathway for oxidation of the reduced phosphorus (P) compound phosphite was cloned from Pseudomonas and also found in other species of bacteria. The enzyme (designated PtxD) was overproduced in the host Escherichia coli by use of a recombinant system. The enzyme was purified to homogeneity via a two-step affinity protocol and characterized.

Phosphorus plays a central role in the metabolism of all living organisms and is a required nutrient. In addition to its role in innumerable metabolic pathways, it is a component of phospholipids, RNA, DNA, and the principal nucleotide cofactors involved in energy transfer and catalysis in the cell. Despite the ubiquitous role of phosphorus (P) in metabolism, the biochemistry of P-containing compounds is generally considered to be quite simple, consisting almost entirely of phosphate-ester and phosphate-anhydride formation and hydrolysis. Thus, it is not surprising that most phosphorus found in living systems is in the form of inorganic phosphate and its esters. However, there are an increasing number of studies showing biochemical reactions of phosphorus compounds that do not involve the formation or hydrolysis of phosphate-esters and phosphate-anhydrides. Some of these reactions involve compounds in which the phosphorus is at a lower valence and oxidation state, suggesting that previously unsuspected phosphorus redox reactions may be important in the metabolism of this element.

Phosphorus has been widely reported to be a redox conservative element in biological systems, with the sum total of phosphorus biochemistry consisting of the formation and hydrolysis of phosphate-ester and anhydride bonds. These reports imply that reduced phosphorus compounds are not important in living systems and that enzymatically catalyzed redox reactions of phosphorus compounds do not occur; however, an increasing body of evidence indicates that this is not the case. Although it is true that inorganic phosphate (+5 valence) is the principal form of phosphorus in living systems, and that phosphate-esters play a critical role in phosphate biochemistry, it is now clear that reduced phosphorus compounds of both natural and xenobiotic origin play important roles in numerous biological systems. Accordingly, many organisms have been shown to possess metabolic pathways for reduction of phosphate to a variety of reduced phosphorus compounds; others have been shown to possess metabolic pathways for oxidation of reduced phosphorus compounds. Among the most striking of these is a recently isolated sulfate-reducing bacterium that obtains all of the energy it requires for growth from the oxidation of phosphite (+3 valence) to phosphate.

Unfortunately, detailed studies examining the mechanisms of biological phosphorus oxidation and reduction are scarce. This is particularly true with regard to the biochemical characterization of putative enzymes involved in reduced phosphorus metabolism. Cell culture studies have shown that certain prokaryotes and eukaryotes oxidize phosphite to phosphate. In addition, cell culture studies have shown the oxidization of hypophosphite (+1 valence) to phosphate.

A few of the enzymes involved in the biosynthesis of the reduced phosphorus antibiotic bialaphos, as well as the enzyme phosphoenolpyruvate phosphonomutase from Tetrahymena, have been purified and characterized. However, these carbon-phosphorus bond-synthesizing enzymes catalyze phosphorus reduction indirectly via intramolecular rearrangements; they do not catalyze direct redox reactions of phosphorus moieties. A similar situation exists for most enzymes involved in carbon-phosphorus bond cleavage. The electron-withdrawing nature of the β-carbonyl groups in phosphonoacetate and phosphonoacetaldehyde renders the carbon-phosphorus bond in each of these compounds susceptible to hydrolytic cleavage by the enzymes phosphonoacetate hydrolase and phosphonoacetaldehyde hydrolase, respectively. Finally, the mechanism of the broad substrate specificity enzyme carbon-phosphorus C-P lyase probably does not involve a simple hydrolytic mechanism, based on the examination of various substrates and their products. However, the mechanism of this enzyme yet remains obscure because in vitro activity of the enzyme has not been achieved, despite numerous attempts, and the identification and characterization of the genes that encode it are also unknown.

Two biochemical studies of putative enzymes that possibly catalyze direct phosphorus redox reactions have been reported using cell suspension studies and partially purified NAD-dependent phosphite oxidoreductase from Pseudomonas fluorescens 195. Cell suspension studies were also reported with a partially-purified hypophosphite oxidase from Bacillus caldolyticus. Although these studies demonstrate the enzymatic nature of the process, they do not greatly add to our understanding of the biochemistry of phosphorus redox reactions because an enzyme that catalyzes a direct phosphorus redox reaction had not been biochemically characterized in pure form prior to work by the inventors.

Pseudomonas stutzeri WM88, capable of oxidizing phosphite and hypophosphite to phosphate, was isolated as a cell suspension (FIGS. 1-4). Molecular and genetic analyses suggested that oxidation of hypophosphite to phosphate in this organism occurs through a phosphite intermediate. These analyses also showed that there are two distinct chromosomal loci responsible for these oxidations: ptxABCD, required for phosphite oxidation, and htxABCDE, required for hypophosphite oxidation.

Oxidoreductases can be used for the synthesis of chiral compounds, complex carbohydrates, and isotopically-labeled compounds. However, these enzymes usually employ cofactors such as reduced nicotinamide adenine dinucleotide (NADH) and nicotinamide adenine dinucleotide phosphate (NADPH). These cofactors are required in stoichiometric amounts with respect to the desired product and are oxidized in the enzymatic reaction producing NAD or NADP. Because the cofactors are expensive, inexpensive methods for their regeneration are highly desirable. Many methods have been employed for cofactor regeneration, such as enzymatic, electrochemical, chemical, photochemical, and biological approaches. Currently, the preferred method for cofactor regeneration involves the use of enzymes known as dehydrogenases that catalyze the oxidation of inexpensive substrates coupled to the reduction of NAD and NADP (EQ. 1). Examples in common use today include formate dehydrogenase (FDH) and glucose dehydrogenase (GDH). The utility of these enzymes for cofactor regeneration is governed by (1) the thermodynamic driving force of the regenerative reaction, (2) the catalytic efficiency of the enzyme, (3) the stability of the enzyme, (4) the cost of producing the enzyme, and (5) the cost of the substrate for the regenerating enzyme.

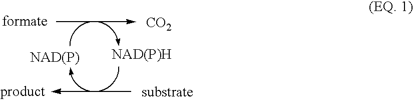

Enzymatic cofactor regeneration is used to regenerate reduced NADH. Advantages of enzymatic strategies for cofactor regeneration include high selectivity, compatibility with the synthetic enzymes, and high turnover numbers. The efficiency of a regenerative system is determined by the expense and stability of the regenerative enzyme and its substrate, the ease of product purification, the catalytic efficiency of the regenerative enzyme (k_{cat}/K_{M}), the K_{M} of the regenerative enzyme for e.g. NAD⁺ and its reduced substrate, and the thermodynamic driving force of the regenerative enzyme.

### SUMMARY OF THE INVENTION

A purified enzyme phosphite dehydrogenasecatalyzes a direct phosphorus redox reaction. Phosphite dehydrogenases are useful for the regeneration of reduced nucleotide cofactors, such as NADH and NADPH, and for oxidizing phosphite to phosphate. In some embodiments, the reduction is performed stereoselectively. In other embodiments, the reduction is performed with an isotope of hydrogen, such as deuterium or tritium.

In one aspect, the phosphite dehydrogenase is a PtxD isolated from organisms such as, but not limited to, Pseudomonas stutzeri WM88, Accession: AF061070; Klebsiella pneumonia, Accession: NC002941; Ralstonia metallidurans; Nostoc punctiforme, Accession: ZP_{—}00110436; Nostoc sp. PCC 7120 plasmid pCC7120gamma, Accession: BAB77417; or Trichodesmium erythraeum IMS101, Accession: ZP_{—}00071268. The phosphite dehydrogenase enzymes described herein may be characterized by each including a common sequence GWRPQFYSLGL. In addition, the phosphite dehydrogenase enzymes described herein include a NAD binding sequence comprising GMGALGKAIAGRL. In addition, the phosphite dehydrogenase enzymes described herein may be characterized by catalytic residues including histidine, glutamate, and arginine.

In another embodiment, the enzyme is prepared from natural sources, and in other embodiments the enzyme is prepared from recombinant processes. In aspects of either embodiment, the enzyme is illustratively purified to 90% purity or greater, to 95% purity or greater. In other aspects, the enzyme is purified to homogeneity.

In another embodiment, a method of purifying a phosphite dehydrogenase is described. The method includes the steps of:

contacting a solution of the enzyme with a first NAD affinity column incapable of binding the enzyme, and eluting the enzyme as a solution having fewer impurities; and

contacting the resulting eluent with a second NAD affinity column capable of binding the enzyme, and eluting the enzyme as a solution.

The second NAD affinity column may be characterized by attachment of the ligand at N-6. The first NAD affinity column may be characterized by attachment of the ligand at C-8.

In another embodiment, a method of preparing NADH or NADPH is described. The method includes the steps of:

contacting a solution of NAD or NADP with a phosphite dehydrogenase and phosphite.

In one aspect, the method of reducing NADH or NADPH includes reducing with an isotope of hydrogen, such as deuterium or tritium, and includes the steps of:

contacting a solution of NAD or NADP with a phosphite dehydrogenase and phosphite, where the phosphite includes the isotope of hydrogen.

In another embodiment, a method of oxidizing phosphite to phosphate is described. The method includes the steps of:

contacting a solution of phosphite with a phosphite dehydrogenase and an oxidizing agent selected from the group consisting of NAD and NADP.

In another embodiment, a method of selectively oxidizing phosphite to phosphate is described. The method includes the steps of:

contacting a solution of phosphite with a phosphite dehydrogenase and an oxidizing agent selected from the group consisting of NAD and NADP, where the solution of phosphite contains at least one other oxidizable species.

In one aspect, the other oxidizable species is selected from the group consisting of hypophosphite, methylphosphonate, arsenite, sulfite, and nitrite.

This invention also describes a purified enzyme phosphite dehydrogenase, useful for the regeneration of reduced nucleotide cofactors, such as NADH and NADPH, for use by other enzymes in enzyme-mediated synthesis. In some embodiments, the enzyme-mediated synthesis is performed stereoselectively. In other embodiments, the enzyme-mediated synthesis is performed with an isotope of hydrogen, such as deuterium or tritium.

In one embodiment, a method of reducing a compound to an overall lower oxidation state is described. The method includes the steps of:

contacting the compound with a first oxidoreductase enzyme that uses a cofactor selected from the group consisting of NADH and NADPH; and

contacting the compound with a phosphite dehydrogenase, phosphite, and an agent selected from the group consisting of NAD and NADP.

In another embodiment, a method of reducing a compound to an overall lower oxidation state, where the reduction includes introducing an isotope of hydrogen, such as deuterium or tritium, is described. The method includes the steps of:

contacting the compound with a first oxidoreductase enzyme that uses a cofactor selected from the group consisting of NADH and NADPH; and

contacting the compound with a phosphite dehydrogenase, phosphite, and an agent selected from the group consisting of NAD and NADP, where the phosphite includes an isotope of hydrogen.

In one aspect, the cofactor is NADH, and the agent is NAD.

In another embodiment, a method of stereoselectively reducing a prochiral compound to an overall lower oxidation state is described. The method includes the steps of:

contacting the prochiral compound with a mixture comprising (a) an oxidoreductase enzyme that uses a cofactor selected from the group consisting of NADH and NADPH, and (b) a phosphite dehydrogenase, phosphite, and an agent selected from the group consisting of NAD and NADP; where the compound is reduced at the prochiral center to form a chiral compound, and a solution of the chiral compound is optically active.

In another embodiment, a method of stereoselectively reducing a prochiral compound to an overall lower oxidation state, where the reduction includes introducing an isotope of hydrogen, such as deuterium or tritium, is described. The method includes the steps of:

contacting the prochiral compound with a mixture comprising (a) an oxidoreductase enzyme that uses a cofactor selected from the group consisting of NADH and NADPH, and (b) a phosphite dehydrogenase, phosphite, and an agent selected from the group consisting of NAD and NADP; where the phosphite includes the isotope of hydrogen; and the compound is reduced at the prochiral center to form a chiral compound, and a solution of the chiral compound is optically active.

In one aspect of the above-described embodiments, the oxidoreductase enzyme is selected from enzymes including, but not limited to, formate dehydrogenase, glucose dehydrogenase, L-lactate dehydrogenase, D-lactate dehydrogenase, malate dehydrogenase, horse liver alcohol dehydrogenase, leucine dehydrogenase, and aldehyde dehydrogenase. It is appreciated that other dehydrogenases that use the cofactors NADH or NADPH are useful in the processes described herein. In another aspect, the cofactor is NADH, and the agent is NAD.

### BRIEF DESCRIPTION OF THE DRAWINGS

FIG. 1 shows structures of the broad-host-range plasmids pWM263 and pWM265 and the physical maps of the cloning vectors pWM263 and pWM265. The large number of unique restriction sites in these plasmids greatly facilitates subcloning of DNA inserted into these vectors. Only unique restriction sites are shown. Two additional plasmids, pWM264 and pWM266, are similar but with the polylinker in the orientation opposite to that in pWM263 and pWM265, respectively. Genes cloned into pWM263 and pWM264 can be expressed from the tac promoter (ptac) in an IPTG (isopropyl-β-D-thiogalactopyranoside)-dependent manner. The rrnB terminator (trrnB) in these plasmids terminates transcripts originating at ptac. All four plasmids can be mobilized to a variety of recipients from E. coli hosts that carry the tra genes of RP4 in trans. The bla gene encodes resistance to β-lactam antibiotics in pWM263 and pWM264. The tetA gene encodes resistance to tetracycline in pWM265 and pWM266. The lacZa gene of pWM265 and pWM266 is not functional due to stop codons in the large polylinkers of these plasmids.

FIG. 2 illustrates deletion analysis of the hypophosphite- and phosphite-oxidizing functions encoded by the plasmid pWM239. A series of deletion derivatives of the plasmid pWM239 were constructed and tested for expression of the hypophosphite and phosphite oxidation phenotypes in E. coli S17-1 and P. aeruginosa PAK Dpil rif: The ability to confer growth in 0.4% glucose-MOPS medium containing hypophosphite (Hpt) or phosphite (Pt) as the sole phosphorus source is indicative of the ability to oxidize the indicated compound to phosphate. Examination of the P oxidation phenotypes displayed by P. aeruginosa carrying the various deletion plasmids indicates that the shaded region between the KpnI and AseI sites is required for Pt oxidation. Further, oxidation of hypophosphite proceeds via a phosphite intermediate. Thus, P. aeruginosa strains carrying plasmids lacking the Pt region are also defective in hypophosphite oxidation. The ability to oxidize Pt in E. coli hosts, is not related to the plasmids, because E. coli is a natural phosphite oxidizer. Therefore, deletions of the Pt region do not affect hypophosphite oxidation in E. coli, and the minimal region required for this phenotype can be determined by examination of the complementation pattern in this host. Accordingly, the shaded region between the SstI and NheI sites is required for hypophosphite oxidation in E. coli. The thick line at the top represents the cloned region of the P. stutzeri chromosome, with the restriction sites used for construction of individual deletions shown. The flanking restriction sites used for construction of each deletion were provided by the polylinker of the vector pWM265 (FIG. 1). The thin lines represent the remaining insert region of each deletion plasmid. Not all restriction sites within the insert were mapped for each enzyme; therefore, the sites shown are not necessarily unique.

FIG. 3 shows P. stutzeri WM88 chromosomal mutations in the region linked to P oxidation phenotypes. The regions of the P. stutzeri WM88 chromosome putatively required for oxidation of hypophosphite (Hpt) and phosphite (Pt) (arrows) were identified by complementation of heterologous hosts. Deletion and insertion mutations in clones of this genomic region were constructed in vitro and recombined onto the P. stutzeri WM88 chromosome as described herein. The P oxidation phenotypes of these mutants were examined by scoring the ability to grow on media containing either Hpt or Pt as the sole P source. These phenotypes confirm that the genes carried in this region are responsible for the oxidation of phosphite and hypophosphite by the original isolate. The thin line at the top indicates the chromosomal region of P. stutzeri under study, with relevant restriction sites shown. Not all sites for each enzyme have been mapped; therefore, the sites shown are not necessarily unique. The thick lines below this represent the extents of the in vitro-constructed deletion mutations. The triangles show the sites of insertion mutations.

FIG. 4 illustrates physical structures of DNA fragments required for oxidation of phosphite and hypophosphite by P. stutzeri WM88. The complete DNA sequences of both fragments were determined as described herein. (GenBank Accession No. AF061070 and AF061267). (A) Structure of a 5.6-kbp KpnI fragment encoding functions required for oxidation of phosphite to phosphate in P. stutzeri WM88. Seven ORFs, indicated by arrows, were identified within this sequence. The ptxE gene is truncated in this clone, as indicated by the partially shaded arrow. Five of these genes, designated ptxA through ptxE, are likely to be involved in oxidation of phosphite and probably form a single transcriptional unit. PtxABC is likely a binding-protein-dependent transport system for the uptake of phosphite. PtxD is an NAD⁺-dependent phosphite dehydrogenase, and PtxE is likely a transcriptional regulator for the ptxABCDE operon. (B) Structure of an 8.9-kbp SstI-to-NheI fragment encoding functions required for oxidation of hypophosphite to phosphite in P. stutzeri WM88. Nine ORFs, indicated by arrows and designated htxA through htxI, are likely to form a single transcriptional unit. Relevant restriction sites used for various plasmid constructions are shown. The BglII and AgeI sites shown in boldface were used as insertion sites for gene disruption experiments (FIG. 3). HtxA is a putative α-ketoglutarate-dependent hypophosphite dioxygenase. HtxBCDE comprise a putative binding-protein-dependent hypophosphite transporter. The remaining genes encode subunits of a putative carbon-phosphorus C—P lyase but are not required for oxidation of hypophosphite. The partially shaded arrow indicates that the htxI gene is truncated in this sequence. Approximately 15 kbp separates the two regions. This 15-kbp region is not required for oxidation of either compound and was not characterized.

FIG. 5 shows overexpression and purification of recombinant PtxD. Protein samples from various stages of the purification were separated by SDS-PAGE and stained with Coomassie Blue. A two step affinity protocol yields homogeneous recombinant enzyme. Lanes 1 and 9, marker proteins (size in kDa is shown); lane 2, lysed cells before IPTG induction; lane 3, lysed cells after IPTG induction; lane 4, crude cell extract; lane 5, cell-free crude extract; lane 6, high speed supernatant; lane 7, flow-through from first NAD affinity column; lane 8, purified enzyme (4.5 μg) from second NAD affinity column.

FIG. 6 shows native gel stained for PtxD activity. PtxD was separated by nondenaturing gel electrophoresis in a 6% continuous gel in HEPES/imidazole buffer. The gel was cut into three identical slices and stained either for total protein or for enzymatic activity. Total protein was detected by staining with Coomassie Blue (lane 1). To detect phosphite-dependent NAD reduction, gel slabs were incubated in Tris buffer with phosphite, NAD, and nitro blue tetrazolium. Production of NADH was detected by precipitation of the reduced tetrazolium dye as a purple band (lane 2). To detect phosphate production, gel slabs were incubated in Tris buffer with phosphite, NAD, and CaCl₂ and stained with methyl green as described herein. Production of phosphate is indicated by a green stained band of precipitated CaHPO (lane 3). A single band is seen in each lane, indicating that a homogeneous preparation of PtxD catalyzes production of phosphate and NADH from phosphite and NAD.

FIG. 7 shows characterization of PtxD with respect to temperature, pH, and salt concentration. A, PtxD activity was assayed in the presence of 20 mM MOPS, pH 7.25, 1 mM phosphite, 0.5 mM NAD, and 10 mM/ml bovine serum albumin at increasing temperatures; B, PtxD activity was assayed in the presence of a 100 mM Tris, 50 mM acetate, 50 mM MES buffer at different pH (adjusted with HCl or NaOH), 1 mM phosphite, and 0.5 mM NAD; C, PtxD activity was assayed in the presence of 20 mM MOPS, pH 7.25, 1 mM phosphite, 0.5 mM NAD, and increasing concentrations of NaCl. The results shown are the average of three experiments.

FIG. 8 shows the initial velocity patterns with NAD and phosphite. The reaction was initiated by adding 3.5 μg of PtxD to the reaction mixture. Left, the concentration of phosphite was varied at the fixed concentrations of NAD. Right, the concentration of NAD was varied at the fixed phosphite concentrations. Concentrations used for both substrates were 45 (●), 56 (□), 71 (◯), 100 (▴), 167 (▪), and 500 (♦)μm. Duplicate assays were performed at each concentration. The curve fits shown represent linear regression analysis of the data from each fixed concentration. Model fitting using the entire data set is described herein, as shown in Tables 3 and 4.

FIG. 9 shows the initial velocity patterns in the presence of the product NADH. The reaction was initiated by adding 3.5 μg of PtxD to the reaction mixture. NADH was included in the assay mixtures at concentrations of 0 (♦), 25 (▪), 50 (▴), 75 (◯), and 100 (□)μm. Left, NAD was held constant at 50 μm with phosphite varied. Right, phosphite was held constant at 50 μm with NAD varied. Duplicate assays were performed at each concentration. The curve fits shown represent linear regression analysis of the data from each fixed NADH concentration. Model fitting using the entire data set is described herein and shown in Tables 5 and 6.

FIG. 10 shows the initial velocity patterns in the presence of the dead end inhibitor sulfite. The reaction was initiated by adding 3.5 μg of PtxD to the reaction mixture. Sulfite was included in the assay mixtures at concentrations of 0 (▴), 5 (▪), 10 (▴), 15 (◯), 20 (□), 25 (●), and 30 (Δ)μm. Left, NAD was held constant at 50 μm with phosphite varied. Right, phosphite was held constant at 50 μm with NAD varied. Duplicate assays were performed at each concentration. The curve fits shown represent linear regression analysis of the data from each fixed sulfite concentration. Model fitting using the entire data set is described herein and shown in Tables 7 and 8.

FIG. 11 shows possible chemical mechanisms for the PtxD reaction. Three possible chemical mechanisms for the concomitant oxidation of phosphite and reduction of NAD are shown. Schemes 1 and 2 involve initial nucleophilic attack at the phosphorus center and subsequent loss of the hydride. Scheme 3 involves initial loss of the hydride to produce the unstable intermediate metaphosphate.

FIG. 12 shows the alignment of PtxD with D-hydroxyacid NAD-dependent dehydrogenases. The amino acids are indicated by their single letter abbreviations. FastA searches with PtxD against the nonredundant Swiss Protein Database show that PtxD is highly homologous to members of the D-hydroxyacid NAD-dependent dehydrogenases family (26-34.5% identical to the top 50 matches, most of which are known or putative members of the family). Representatives (crystal structures are available for five of the six sequences used) from this family were aligned with PtxD using Clustal W (Thompson et al., 22 Nucleic Acids Res. 4673-4680 (1994), the disclosure of which is incorporated herein by reference), showing conservation of important features. Solid arrow, the NAD binding motif; asterisks, the putative catalytic residues; dashed arrow, a conserved signature sequence for the D-isomer-specific 2-hydroxyacid family. Proteins used were as follows. PtxD, phosphite dehydrogenase from P. stutzeri WM88; FDH, formate dehydrogenase from Pseudomonas sp. 101; LDH, D-lactate dehydrogenase from Lactobacillus helveticus; GDH, D-glycerate dehydrogenase from Hyphomicrobium methylovorum GM2; SerA, D-3-phosphoglucerate dehydrogenase from E. coli; PdxB, erythronate-4-phosphate dehydrogenase from E. coli; HICDH, D-2-hydroxyisocaproate dehydrogenase from Lactobacillus casei. Swiss Protein accession numbers for the sequences used are O69054, P33160, P30901, P36324, P08328, P05459, and P17584, respectively.

FIG. 13 shows ¹H NMR spectra of (A) commercial NADH, (B) (4S)-[4-²H]-NADH, (C) (4R)-[4-²H]-NADH, and (D) the product formed by incubation of PtxD with ²H-phosphite.

FIG. 14 shows reciprocal plots of the initial rates in the reaction of PtxD with unlabeled phosphite (squares, ▪, □) and deuterium-labeled phosphite (circles, ◯, ●) at fixed NAD⁺ concentrations of 165 (closed symbols) and 500 μM (open symbols).

FIG. 15 shows cofactor regeneration of PtxD and LLDH, varying the ratio of the enzymes (▪, 2:1 U LLDH:PtxD; and ●, 1:2 U LLDH:PtxD; 1:40 NAD⁺:synthetic substrate) and the loading of the catalytic NAD⁺ (▴1:400 NAD⁺:synthetic substrate).

FIG. 16 shows cofactor regeneration with PtxD and LLDH, monitored by ¹H or ³¹P NMR spectroscopy.

FIG. 17 shows two separate runs (◯ and □) using PtxD for cofactor regeneration with LLDH.

FIG. 18 is a titration curve used to monitor the amount of NADH in solution upon addition of PtxD to a solution containing 0.6 U HLADH, 200 mM phosphite, 100 mM acetaldehyde, and 0.1 mM NAD.

FIG. 19 shows ¹H NMR spectra of unlabeled L-lactic acid (top) and a solution of [2-²H]-L-lactic acid prepared from deuterium labeled phosphite.

FIG. 20 is a PtxD protein with an amino acid sequence as shown for alcaligenes, a nucleotide sequence of the DNA encoding the protein. The amino acids are indicated by their single letter abbreviations. The protein sequence from Alcaligenes faecalis is about 50% identical to the published sequence from Pseudomonas stutzeri. This protein was purified as a fusion to maltose binding protein using the pMal system from New England Biolabs, and it has an activity level comparable to PtxD from Pseudomonas stutzeri.

FIG. 21 is a PtxD protein with an amino acid sequence as shown for xanthobacter, and a nucleotide sequence of the DNA encoding the protein. The amino acids are indicated by their single letter abbreviations. The protein from Xanthobacter flavus molecular sequence is about 50% identical to the published sequence from Pseudomonas stutzeri. It can be overexpressed in E. coli and it has an activity comparable to PtxD from Pseudomonas stutzeri in cell extracts.

FIG. 22 shows amino acid sequences and nucleotide sequences for 3 of the four proteins (WM1639, WM1686, WM1733, WM2048). The amino acids are indicated by their single letter abbreviations. The amino acid sequences are virtually identical (˜98%) to the published sequence from Pseudomonas stutzeri; (a) is an amino acid sequence from an organism most closely related to Pseudomonas putida, WM1639; (b) is an amino acid sequence from an organism most closely related to Klebsiella ornithinolytica, WM1686 and a nucleotide sequence of the DNA encoding the protein; (c) is an amino acid sequence from an organism most closely related to Klebsiella oxytoca, WM1733 and a nucleotide sequence of the DNA encoding the protein; (d) is an amino acid sequence from an organism most closely related to Pseudomonas stuzeri, WM2048 and a nucleotide sequence of the DNA encoding the protein.

FIG. 23 shows amino acid sequences of PtxD homologs that were located in the sequence databases: 23(a) shows a sequence of a protein from Nostoc punciforme); 23(b) shows a sequence of a protein from Nostoc PCC1720; 23(c) shows a sequence of a protein from Tricodesmium; 23(d) shows a sequence from a protein from Ralstonia. The amino acids are indicated by their single letter abbreviations. These homologs include one from Nostoc PCC1720 that was overexpressed and shown to possess phosphite oxidation activity. This shows that using the present invention, functionally similar proteins will be readily found elsewhere by those of skill in the art.

FIG. 24 shows a sequence alignment of phosphite dehydrogenases derived from Pseudomonas stutzeri, Alcaligenes faecalis, Nostoc PCC1720, Xanthobacter flavus, Nostoc punctiformae, Tricodesmium erythraeum, Ralstonia metallidurans, Klebsiella pneumonia, Pseudomonas putida (WM1639), Klebsiella ornithinolytica (WM1686), Klebsiella oxytoca (WM1733), and Pseudomonas stuzeri (WM2048). The alignment was generated using Clustal W (1.82) (Thompson et al., 22 Nucleic Acids Res. 4673-4680 (1994), the disclosure of which is incorporated herein by reference). The amino acids are indicated by their single letter abbreviations. The symbol (*) denotes a conserved amino acid; and the symbol (:) denotes a functionally-equivalent amino acid, such as those equivalents described in Dayhoff matrices.

### DETAILED DESCRIPTION OF THE INVENTION

A gene encoding an enzyme required for operation of a novel biochemical pathway for oxidation of phosphite, a reduced phosphorus (P) compound, was cloned from Pseudomonas and also identified in other bacteri. The enzyme (designated PtxD) was overproduced in the host Escherichia coli by use of a recombinant system. The enzyme was purified to homogeneity via a two-step affinity chromatography protocol and characterized. The enzyme stoichiometrically produces NADH and phosphate from NAD and phosphite, respectively. Mechanistic studies indicate stereoselective transfer of hydride from phosphite to the Re-face of NAD⁺ with observed steady-state kinetic isotope effects of 2.1 on Vₘₐₓ and 1.8 on Vₘₐₓ/K_{M}.

Oxidation of phosphite occurs by the action of an NAD-dependent phosphite dehydrogenase activity encoded by the ptxD gene. PtxD is highly homologous to a large number of proteins of this general type, most notably those involved in the oxidation of 2-ketoacids. PtxD exhibits 27 to 33% identity to various members of the family, including conservation of the NAD binding site and important catalytic residues.

Biological redox reactions involving phosphorus compounds are poorly understood, at best, due to a dearth of biochemically characterized enzymes. PtxD, an enzyme that catalyzes oxidation of the reduced inorganic phosphorus compound phosphite, was purified to homogeneity. An aspect of the invention is an enzyme in a pure form that catalyzes direct oxidation of a reduced phosphorus compound.

The ptxD gene from Pseudomonas stutzeri WM88 encoding the novel phosphorus oxidizing enzyme NAD:phosphite oxidoreductase (trivial name phosphite dehydrogenase, PtxD) was cloned into an expression vector and overproduced in Escherichia coli. The heterologously produced enzyme is comparable to the native enzyme based on mass spectrometry, amino-terminal sequencing, and specific activity analyses.

Recombinant PtxD was purified to homogeneity via a two-step affinity protocol and characterized. The enzyme stoichiometrically produces NADH and phosphate from NAD and phosphite. The reverse reaction, where phosphate and NADH are converted into phosphite and NAD, respectively, was not observed. Gel filtration analysis of the purified protein is consistent with PtxD acting as a homodimer. PtxD has a high affinity for its substrates with K_{M} values of 53.1±6.7 μM and 54.6±6.7 μM for phosphite and NAD, respectively, Vₘₐₓ and k_{cat} were determined to be 12.2±0.3 μmol min⁻¹ mg⁻¹ and 440 min⁻¹. NADP can substitute for NAD in the oxidation of phosphite; however, NADP has a higher K_{M}. In contrast, none of the numerous other compounds examined were able to substitute for phosphite in the enzymatic conversion. Initial rate studies in the absence or presence of products, and in the presence of the dead end inhibitor sulfite are most consistent with a sequential ordered mechanism for the PtxD reaction, with NAD binding first and NADH being released last. Amino acid sequence comparisons place PtxD as a new member of the D-2-hydroxyacid NAD-dependent dehydrogenases, the only one to have an inorganic substrate. This was also the first heterologous expression of the protein. The enzyme is capable of direct oxidation of a reduced phosphorus compound.

Mechanistic studies indicate stereoselective transfer of hydride from phosphite to the Re-face of NAD⁺ with observed steady-state kinetic isotope effects of 2.1 on Vₘₐₓ and 1.8 on Vₘₐₓ/K_{M}. The novel enzyme is useful for methods requiring regenerating the cofactor NADH, for use in synthetic oxidoreductases, and to synthesize chiral compounds, complex carbohydrates, and isotopically-labelled compounds. This enzyme is superior to currently available enzymes used for this purpose due to the higher thermodynamic driving force provided by the oxidation of phosphite, the higher activity of the enzyme, and the ability of the enzyme to utilize both NAD and NADP as substrates. Further, substantial improvement of the catalytic efficiency, stability, and thermal properties of the enzyme is contemplated and should be possible using standard molecular biological techniques.

The enzyme PtxD couples the oxidation of phosphite to the reduction of NAD according to the following reaction (EQ. 2):

HPO₃⁻²+NAD⁺+H₂O→HPO₄⁻²+NADH+H⁺ (EQ. 2)

ΔG^{0,}=−63.3 kJ/mol, K_{eq}=1.34×10¹¹ (EQ. 3)

The low free energy (ΔG^{0,}) and high equilibrium constant (K_{eq}) make PtxD vastly superior to all other known regenerating enzymes with respect to thermodynamic driving force (EQ. 3) (for comparison the FDH numbers are ΔG^{0,}=−33.2 kJ/mol, K_{eq}=6.8×10⁵; thus, the PtxD reaction has approximately twice the thermodynamic driving force of FDH resulting in the equilibrium constant being 100,000-fold higher in the direction of NADH formation). PtxD is expected to be comparable to other known regenerating enzymes with respect to each of the other criteria.

A general overview of cofactor regeneration is shown in EQ. 4. As shown, a synthetic enzyme system is coupled to a regenerative system for the continual replenishment of the reduced nicotinamide cofactor. After the reactant for the synthetic system is exhausted, the desired product is isolated from the reaction mixture. In addition to reducing the cost of stereoselective synthesis, cofactor regeneration simplifies product isolation, and prevents problems of product inhibition of the synthetic enzyme by the cofactor when used stoichiometrically. Moreover, cofactor regeneration influences the position of the equilibrium of the synthetic enzyme system, i.e. the regenerative system may drive the synthetic reaction to completion, even when product formation would be unfavored in the absence of the regenerative system.

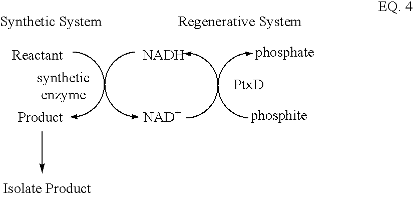

The oxidation of NAD⁺ by phosphite to NADH, with concomitant formation of phosphate, catalyzed by phosphite dehydrogenase (PtxD) has an extremely high thermodynamic driving force of −63.3 kJ/mol resulting in a K_{eq} of 1×10¹¹. The enzyme is used for the efficient regeneration of NADH for use by synthetic oxidoreductases. The efficiency of PtxD for cofactor regeneration was determined with L-lactate dehydrogenase, D-lactate dehydrogenase, horse liver alcohol dehydrogenase, and aldehyde dehydrogenase with total turnover numbers of NAD⁺ up to 2000 and total turnovers up to 10⁵ for PtxD. Phosphite dehydrogenase was also used for the synthesis of [2-²H]-L-lactic acid from deuterated phosphite, demonstrating the potential of the process for stereoselective preparation of isotopically labeled compounds (Vrtis et al., Angew. 41 Chem. Intl. Ed. Engl. 3257-3259 (2002), the disclosure of which is incorporated herein by reference).

In practice the invention calls for setting up a reaction mixture containing an enzyme catalyzing the desired reduction and its starting substrate, PtxD and its substrate phosphite, and a small amount of NAD(P). NAD(P)H produced by the PtxD reaction will serve as substrate for the second enzyme. During reaction of the second enzyme with its substrate and NAD(P)H, the desired product will be produced along with NAD(P). The cycle will then be repeated until the desired substrate or phosphite (or both) is exhausted (see EQ. 4).

The final amount of the desired product is governed by the ratio of thermodynamic driving forces of the phosphite/phosphate and substrate/product reactions. The very high driving force provided by the PtxD will ensure that a typical reaction of substrate to product will go to completion (i.e. be very efficient). This high driving force should also allow substrate to product reactions not possible with currently used coupling enzymes due to unfavorable energetics. Finally, it is very likely that the enzyme may be improved by standard molecular methods to produce PtxD derivatives that are superior than existing regeneration systems with respect to the other criteria outlined above.

Enantiomerically pure lactic acid from pyruvic acid is produced in a coupled reaction using lactate dehydrogenase, PtxD, and phosphite. Either D-lactate or L-lactate can be produced depending on whether D-lactate dehydrogenase or L-lactate dehydrogenase is used.

Currently, the enzymes most commonly used for regeneration of NADH include formate dehydrogenase (FDH), glucose dehydrogenase (GDH), and glucose-6-phosphate dehydrogenase (G6PDH). Phosphite dehydrogenase (PtxD) is superior to these proteins with respect to many of the critical requirements for an efficient regenerative enzyme. The low free energy (ΔG^{0,}=−63.3 kJ/mol) and associated high equilibrium constant (K_{eq}=1×10¹¹) for the reduction of NAD⁺ by phosphite assure that NADH regeneration is more strongly driven than in any other enzymatic regeneration system. For comparison, FDH and GDH have equilibrium constants of 7×10⁵ and 5×10³, respectively, for NADH regeneration. The strong driving force should also permit PtxD to catalyze reactions that are thermodynamically unfavorable, e.g it may be used with glucose dehydrogenase, formate dehydrogenase, and aldehyde dehydrogenase, enzymes that catalyze reactions with a ΔG^{0,} of −21.0, −33.2, and −53.6 kJ/mol, respectively. No other regenerative enzyme is capable of driving these processes uphill. In certain cases, the energetics of these enzymes is such that their reactions essentially only go in the direction of oxidizing the substrate and forming NAD(P)H. Driving these enzymes in the reverse direction, namely reducing the oxidized substrate using NAD(P)H, with concomitant formation of NAD(P) is facilitated by a cofactor regeneration enzyme whose reaction is sufficiently energetically favorable. For example, formate dehdyrogenase does not have a sufficient driving force, as illustrated by the relatively low free energy ΔG^{0,}.

Moreover, the K_{M} for NAD⁺ and phosphite are low (˜50 μM), the specific activity of PtxD (12 μmol min⁻¹ mg⁻¹) is larger than that of FDH (3 μmol min⁻¹ mg⁻¹) (Popov et al., 301 Biochem. J. 625-643 (1994); Schutte et al, 62 Eur. J. Biochem. 151-60 (1976), the disclosures of which are incorporated herein by reference), and inhibition by NAD⁺ is less prominent for PtxD (Kᵢ=223 μM) than for FDH (Kᵢ=150 μM). In addition, PtxD can be employed for the synthesis of isotopically-labeled compounds. The cost of preparing the deuterium-labeled phosphite required for the processes described herein is less than that of either deuterated formate or glucose, which are required for preparing labeled products with FDH and GDH, respectively. Phosphite and phosphate should not interfere with separation, isolation, or purification of the synthetic product, and phosphite may be used as the buffer for the system. In addition, phosphate does not act as an inhibitor of PtxD at concentrations as high as 500 mM. However, the other product, NADH, is a competitive inhibitor with respect to both phosphite and NAD at 4 mM.

As described herein, assays were carried out to determine the utility of phosphite dehydrogenase as a regenerative system with the enzymes L-lactate dehydrogenase (LLDH), D-lactate dehydrogenase (DLDH), horse liver alcohol dehydrogenase (HLADH), malate dehydrogenase (MDH), and aldehyde dehydrogenase (ADH). With respect to the results provided herein, TTN for NAD⁺ refers to the total number of moles of product formed per mole of cofactor during the course of a complete reaction (EQ. 5). The TTN for the regenerative enzyme is measured as moles of product formed per mole of enzyme. The turnover number (TN) is defined by the moles of product formed per mole of cofactor (or enzyme) per unit time (EQ. 6).

$$\begin{matrix}TTN = \frac{moles product formed}{( moles cofactor ( or enzyme ) in reaction} & EQ . 5 \\ TN = \frac{moles product formed}{( moles cofactor \times ( time )} & EQ . 6\end{matrix}$$

Unlike most NAD-dependent dehydrogenases, PtxD does not appear to catalyze the reverse reaction (i.e. reduction of phosphate with NADH) to a measurable extent. At pH 7, the reduction potential (E) of the phosphate/phosphite couple is −650 mV, while that of the NADH-NAD couple is −320 mV. Thus, the reduction of NAD by phosphite is a significantly exergonic reaction (ΔG⁰=−63.32 kJ/mol). Using this value, the equilibrium constant for the forward reaction is calculated to be 1.34×10¹¹, and hence, the reduction of NAD by phosphite is essentially irreversible under physiological conditions. While not being bound by theory, it is believed that these thermodynamic relationships account for the observation that PtxD operates as a cofactor regenerating enzyme for applications that require continuous regeneration of NADH, as described herein.

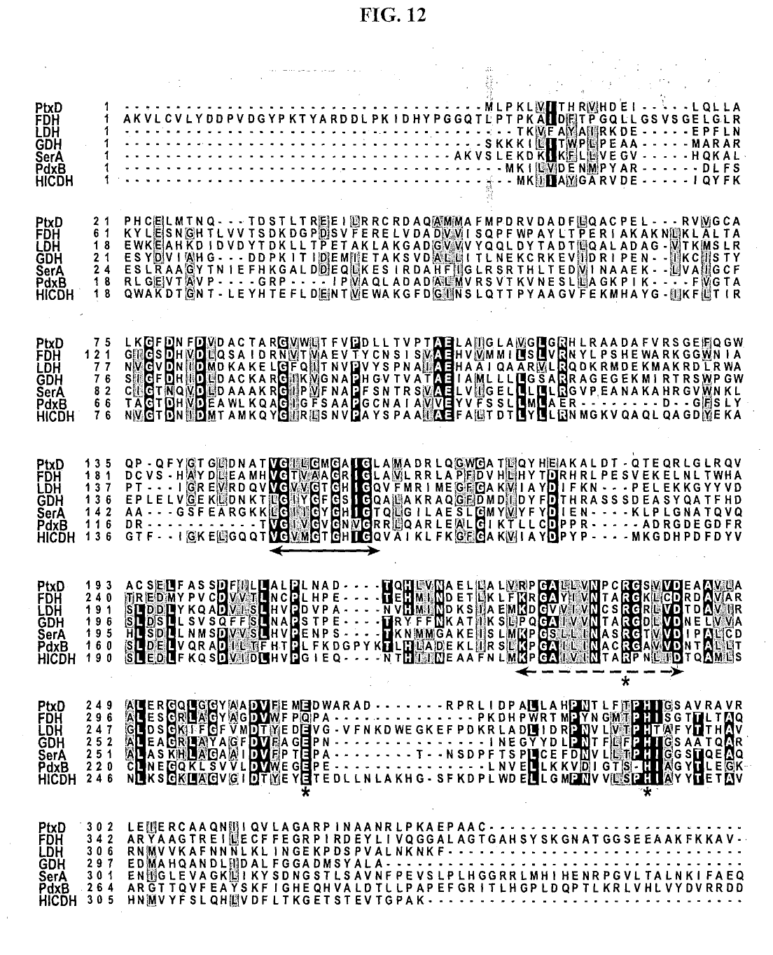

Amino acid sequence comparisons indicate that PtxD is a member of the D-isomer-specific, 2-hydroxyacid NAD-dependent dehydrogenase protein family, the first discovered with an inorganic substrate. An alignment of PtxD with several members of this family shows that it shares many of their characteristics, including the conserved NAD binding site and one of the Prosite signature sequences for this enzyme family (FIG. 12). Chemical modification, site-directed mutagenesis, and crystallographic studies of several D-isomer-specific dehydrogenases have pointed to three residues, His²⁹², Glu²⁶⁶, and Arg²³⁷ (PtxD numbering) essential for catalysis in this family of enzymes. Each of these residues is conserved in the enzyme family, and each is also present in PtxD. Formate dehydrogenase is distinguished in that it has a glutamine residue instead of the glutamate. In addition, these three residues correspond to the catalytic residues His¹⁹⁵, Asp¹⁶⁸, and Arg¹⁷¹ from L-lactate dehydrogenase. Similar to the proposed roles of these residues in lactate dehydrogenase, His²⁹² is believed to act as a proton donor, Glu²⁶⁶ is believed to stabilize the positive charge from the protonated histidine, and Arg²³⁷ is believed to bind the carboxylate moiety of the hydroxyacid. Moreover, in the case of PtxD, it seems plausible that Arg²³⁷ could bind the ionized hydroxyl groups of phosphite.

PtxD has been located in organisms other than Pseudomas stutzeri, Accession: AF061070.

Additional sequences and accession numbers are as follows: Klebsiella pneumonia, Accession: NC002941; Ralstonia metallidurans; Nostoc punctiforme, Accession: ZP_{—}00110436; Nostoc sp. PCC 7120 plasmid pCC7120 gamma, Accession: BAB77417; Trichodesmium erythraeum IMS101, Accession: ZP_{—}00071268.

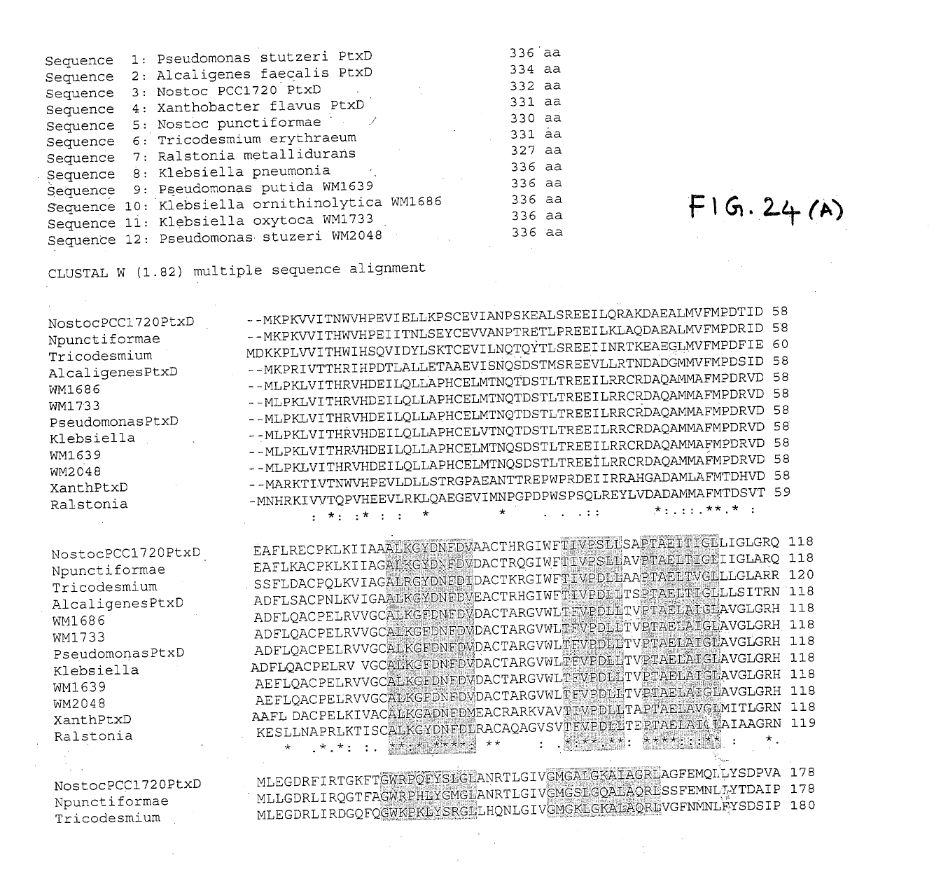

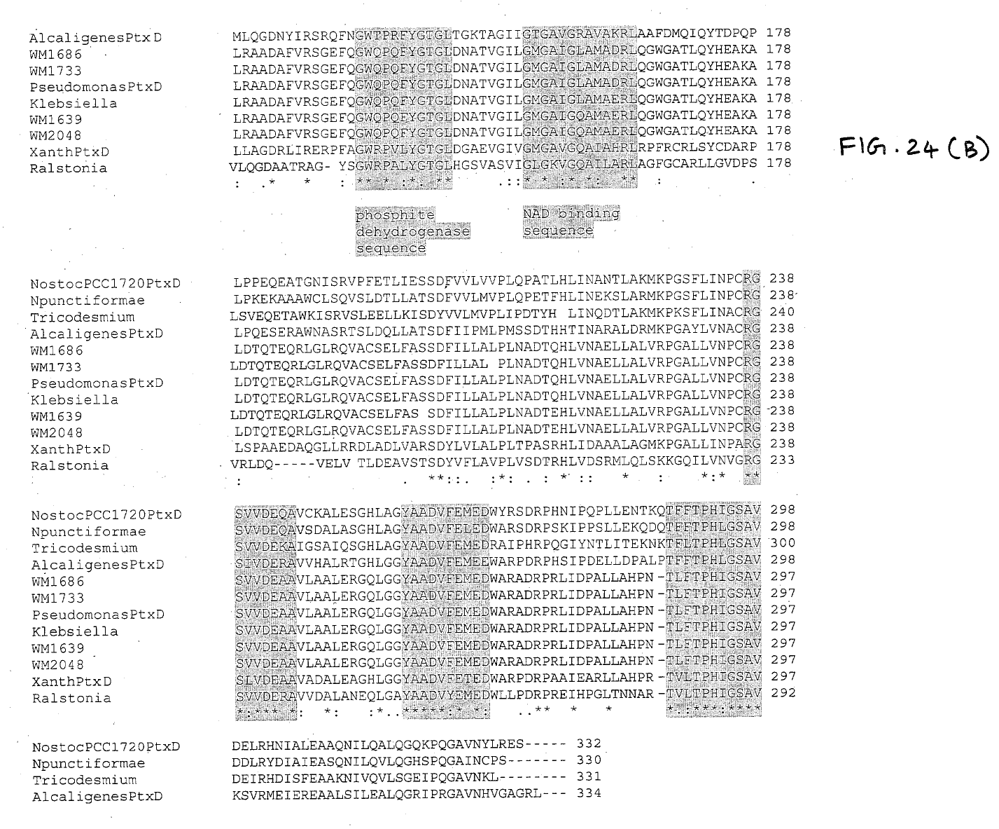

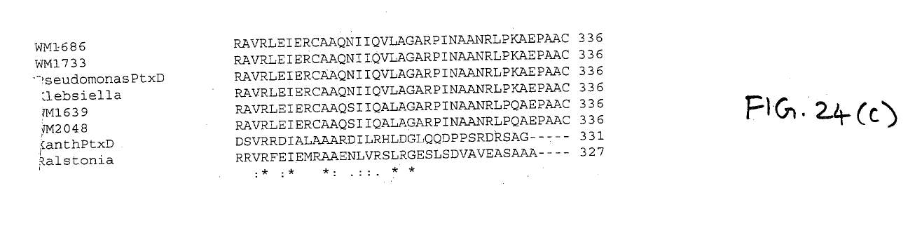

As shown in the sequence alignment illustrated in FIG. 24, the phosphite dehydrogenases that have been isolated are homologous. In addition, certain regions within the enzyme are highly homologous. In particular, the sequence GMGAIGLAMADRL from P. stutzeri PtxD corresponds to the sequence typically attributed to NAD binding. Nevertheless, some variation is this sequence is observed in the examples shown in FIG. 24. Exemplary of this variation is the sequence GX₁GX₂X₃GX₄AX₅X₆X₇RL observed in all the sequences illustrated in FIG. 24, where X₁ is M, T, or L; X₂ is K, S, or A; X₃ is V, I, or L; X₄ is Q, L, R, or K; X₅ is I, M, V, or L; X₆ is L or A; and X₇ is A, H, E, D, K, Q, or G. It is appreciated that all of these sequence variations are capable of recognizing NAD. In addition, it is appreciated that certain amino acid variations represent substitutions that will not substantially affect the binding ability of the enzyme, such as the amino acid variations denoted by (:).

In addition, while not being bound by theory, the sequence GWQPQFYGTGL may be responsible for imparting to the PtxD from P. stutzeri PtxD its ability to use phosphite as a substrate. This sequence also appears in variations, exemplified by the sequence GWX₁PX₂X₃YX₄X₅GL, where X₁ is R, Q, T, or K; X₂ is A, V, Q, R, K, or H;X₃ is L or F;X₄ is G or F; and X₅ is T, R, M, or L.

Other regions of high amino acid homolog are shown in FIG. 24 as grey regions. It is appreciated that the general class of phosphite dehydrogenase enzymes, including the exemplary embodiments included herein, may be described by each of these highly homologous regions.

The ptxD Gene Encodes an NAD:Phosphite Oxidoreductase

The ptxD gene was cloned into a T7 expression plasmid and overexpression of the PtxD protein in E. coli was achieved. Crude cell extracts were prepared from IPTG-induced strains carrying the ptxD overexpression plasmid, pWM302, and from control cells carrying the overexpression vector, pET11a, without an insert. Phosphite-dependent NAD reduction (specific activity ˜0.2 units/mg) was observed in extracts prepared from the PtxD overexpression strain after high speed centrifugation to remove the membrane-associated NADH oxidase activity (high speed extracts). No activity was observed in high speed extracts of the vector only control, indicating that this activity was dependent on the ptxD gene.

Similarly, phosphite-dependent NAD reduction was detected in high speed cell extracts of P. stutzeri WM567 grown in media with either phosphite or hypophosphite as sole phosphorus sources (specific activity ˜0.02 units/mg for both). However, the observed enzyme activity was significantly lower than that observed in extracts of the overproducing E. coli strain. Phosphite dependent NAD reduction (specific activity ˜0.02 units/mg) was also observed in high speed extracts prepared from P. stutzeri WM567 grown in medium with a growth-limiting concentration of phosphate as the sole phosphorus source, while PtxD activity was not detected in extracts of cells grown in medium with excess phosphate. No activity was detected in extracts of the ptxD mutant P. stutzeri WM581 grown in phosphate-limiting medium, which again demonstrates that this activity requires the ptxD gene. Taken together, these data clearly indicate that the ptxD gene encodes an NAD:phosphite oxidoreductase. Further, the data obtained from P. stutzeri extracts indicate that ptxD expression is induced by phosphate starvation.

Purification of Native and Recombinant PtxD

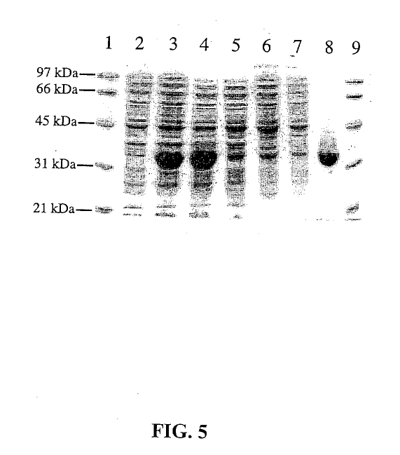

A two-step NAD-affinity protocol was developed that allows purification of recombinant PtxD after overexpression in E. coli. PtxD does not bind an NAD affinity column with C-8 attachment of the ligand. This step is used to reduce the number of other putative NAD-binding enzymes present in the high speed cell extract. PtxD does bind a second NAD affinity column with attachment of the ligand at N-6. This binding occurs even in the presence of 1 M NaCl, which is used to reduce binding of unwanted proteins. An elution gradient of 0-3 mM NAD is used to recover the adsorbed protein from this second column. Other putative NAD-binding enzymes co-elute with PtxD for about half of the elution gradient. These fractions, estimated to be 95% pure, were used for preliminary analyses. During the second half of the elution gradient, the fractions contained homogenous PtxD as shown by SDS-PAGE (FIG. 5). The routine purification yield is ˜50% (Table 1). Native PtxD is purified by the same protocol from extracts of hypophosphite-grown P. stutzeri WM536. As with recombinant PtxD, native PtxD did not bind the first affinity column, but it did bind the second column in the presence of 1 M NaCl. A preparation of native enzyme that had been purified through the tandem affinity protocol twice gave a preparation that was ˜90% pure as estimated by SDS-PAGE, with a yield of 9.3% (Table 1). This latter preparation was sufficiently pure to allow mass spectrometric and amino terminus analyses. The specific activity of the purified recombinant protein is essentially identical to that of the purified native protein.

Mass Spectrometry and Amino Terminus Sequencing

Production of proteins in recombinant hosts does not always produce a suitable protein. To, verify that PtxD produced in E. coli is identical to that produced by the native host, the first 15 residues of the PtxD amino terminus from each preparation were sequenced. Both preparations yielded the sequence MLPKLVITHRVHDEI, which is in complete agreement with the amino acid sequence that may be predicted from the DNA sequence. Mass spectrometry analyses was also carried out to examine whether PtxD is modified in either of the two organisms. The native and recombinant proteins gave peaks of 36,413±18 and 36,430±18 daltons, respectively, in agreement with the predicted molecular mass of PtxD of 36,415 daltons. These results indicate that both organisms produce the same unmodified enzyme. In addition, both samples had an additional peak of approximately similar height corresponding to a mass ˜190 daltons smaller than the predicted molecular mass (36,239±18 daltons for the native preparation and 36,226±18 daltons for the recombinant preparation). Because a unique amino-terminal sequence was obtained from both preparations, the smaller peak likely represents a modified form of PtxD rather than a contaminating protein of nearly identical molecular weight. Further, the unique amino-terminal sequence suggests that the lower molecular weight peak is not the result of amino-terminal processing of PtxD. The loss of the two C-terminal residues (m-AC, 174 daltons) is a possible explanation for this result. A mixture of 50% native and 50% recombinant PtxD gave only the same two peaks, suggesting that the smaller mass peak it is not an artifact of overexpression in E. coli. The recombinant enzyme was used for all of the remaining studies disclosed herein.

Characterization of PtxD

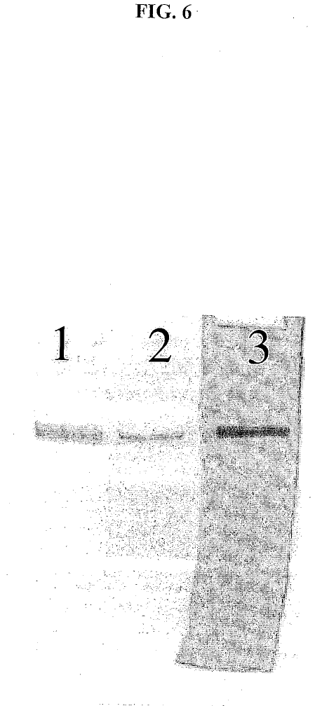

Homogeneous preparations of PtxD catalyze the oxidation of phosphite to phosphate coupled to the reduction of NAD to NADH. Heat-denatured PtxD is incapable of catalyzing phosphite oxidation and NAD reduction. In addition, production of phosphate and NADH was shown to be catalyzed by a single protein using enzymatic activity stains (FIG. 6). When assayed under standard conditions, the specific activity of PtxD, measured independently by the production of either phosphate or NAD, was 10.6 and 10.3 units/mg, respectively, indicating that phosphate and NADH production is stoichiometric. The reverse reaction, as measured by phosphate-dependent NADH oxidation, was not observed (with 4 mM phosphate and 1 mM NADH).

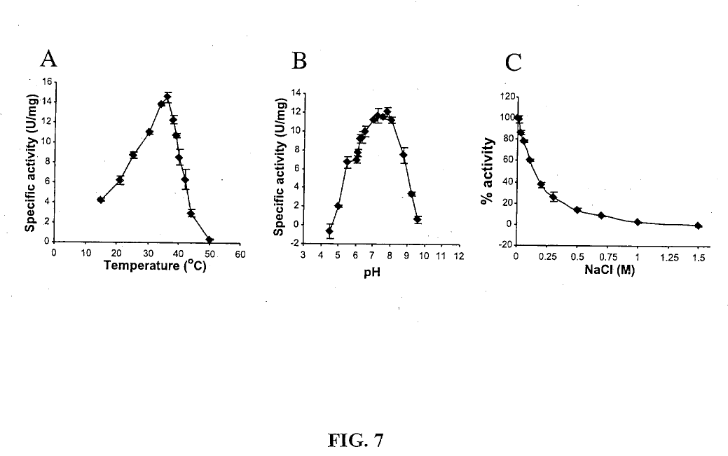

Gel filtration analyses of purified PtxD suggest a native molecular mass of ˜69 kDa, consistent with the enzyme being a homodimer (the predicted molecular mass of the homodimer is 72.8 kDa). PtxD has a temperature optimum of 35° C. with a sharp decrease in activity at higher temperatures (FIG. 7A). It is active through a wide pH range (pH 5-9) with maximum activity from 7.25 to 7.75 (FIG. 7B). The addition of NaCl to the assay buffer has a negative effect on enzyme activity, with only 37% of the activity left at 200 mM NaCl (FIG. 7C). The addition of either EDTA or EGTA (10 mM final concentrations) to the assay buffer has no effect on enzyme activity, indicating that loosely bound metals are not critical to the operation of the enzyme in catalysis.

Several alternative substrates were tested for their ability to substitute for either NAD or phosphite (Table 2). NADP is able to substitute for NAD at higher concentrations and results in reduced rates. None of the compounds tested were able to substitute for phosphite. These tested compounds included several compounds that are substrates for homologous enzymes (glycerate, phosphoglycerate, lactate, 2-hydroxyisocaproate, and formate) and others (hypophosphite, methylphosphonate, arsenite, sulfite, and nitrite) that are structurally or chemically similar to phosphite. The ability of PtxD to utilize alternate substrates in the reverse direction was also explored. As described herein, PtxD is unable to catalyze the reverse reaction (phosphate reduction) using NADH as an electron or hydride donor. PtxD is also unable to catalyze the reduction of nitrate, arsenate, sulfate, acetate, bicarbonate, methylphosphonate, aminoethylphosphonate, glycerate, or pyruvate (potential substrates were tested at 4 mM with 1 mM NADH; the limit of detection is ˜0.025, units/mg under these conditions). However, PtxD did catalyze the reduction of hydroxypyruvate (4 mM hydroxypyruvate, 1 mM NADH), at a low level (0.14 units/mg). As PtxD activity is induced by phosphate starvation, the foregoing implies that the true substrate of PtxD is a phosphorus compound and that the function of PtxD is to provide the cell with an alternate source of phosphorus. Conversely, several of these homologous enzymes were tested for NAD-dependent oxidation of phosphite without any observed activity.

Several substrate analogs were examined for inhibitory activity (Table 2). Sulfite was a strong inhibitor of PtxD activity, while nitrite, formate, D-glycerate, D-2-hydroxy-4-methylvalerate, hydroxyisocaproate, and arsenite moderately inhibited the activity. Several of the cofactor analogs tested were weak enzyme inhibitors, including ATP, ADP, ADP-ribose, and NADP. AMP does not inhibit PtxD. Detailed kinetic studies of enzyme inhibition are described herein.

Initial Rate Studies

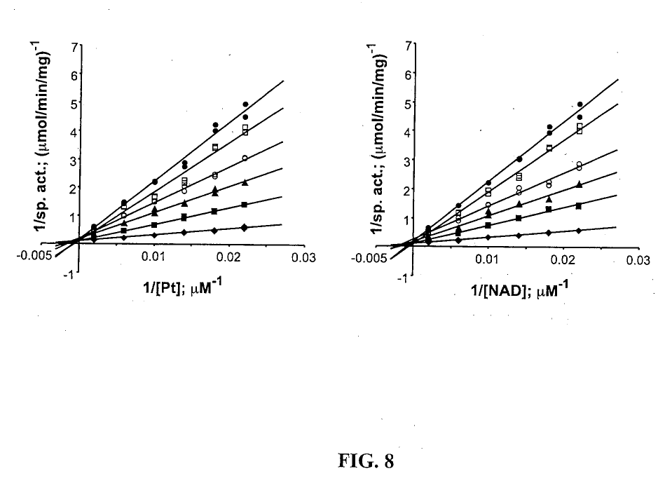

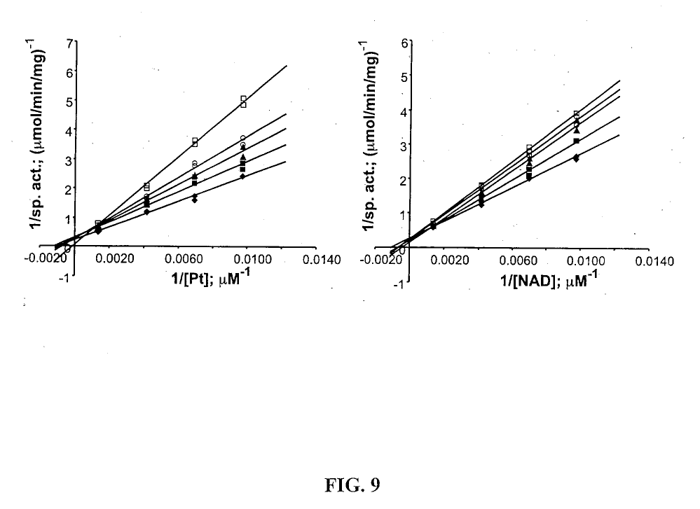

PtxD activity was determined with varying levels of substrates in the absence of products (FIG. 8), and the data were fit to various kinetic models using a modified version (Robertson, J. G. IntelliKinetics, version 1.01, Pennsylvania State University, State College, PA (1991)) of the program of Cleland (63 Methods Enzymol. 103-138 (1979), the disclosure of which is incorporated herein by reference). These initial rate data show that the enzyme follows Henri-Michaelis-Menten kinetics and suggest that the reaction proceeds via a sequential mechanism. The K_{M} values were determined to be 53.1±6.7 and 54±6.7 mM for phosphite and NAD, respectively. The Vₘₐₓ is 12.2±0.3 mmol min⁻¹ mg⁻², and k_{cat} is 440 min⁻¹ (per monomer). Data from fits to the sequential mechanism and to alternative mechanisms are presented in the supplementary material (Tables 3 and 4).

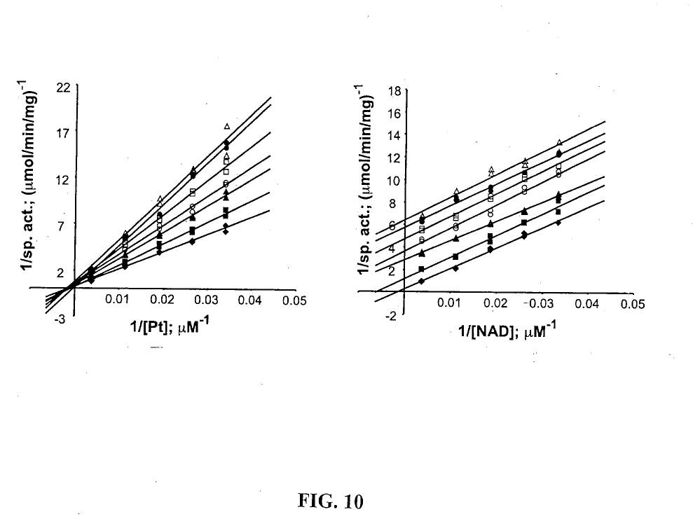

To distinguish between the random and ordered sequential mechanisms, initial rate studies were also carried out in the presence of products and in the presence of the dead end inhibitor sulfite. The type of inhibition and kinetic constants were determined by fitting the data to various kinetic models (Cleland, 63 Methods Enzymol. 103-138 (1979); Cleland, in The Enzymes Vol. 2, 3rd Ed., 1-65, Academic Press, London (Boyer, P. D., ed. 1970), the disclosures of which are incorporated herein by reference). As described herein, phosphate does not inhibit the PtxD reaction at a concentration of 4 mM; therefore, inhibition at higher levels of phosphate was tested. No inhibition of PtxD activity by phosphate was observed with both phosphite and NAD held at concentrations approximating their respective K_{M} values (50 mM each) even at phosphate concentrations of 100 mM. Thus, phosphate does not inhibit the PtxD reaction. In contrast, NADH does inhibit the PtxD reaction (FIG. 9). Initial rate studies in the presence of NADH suggest that it is a competitive inhibitor with respect to both phosphite (Kᵢₛ=115±6 mM) and NAD (Kᵢₛ=233±15 mM). Initial velocity studies in the presence of the dead end inhibitor sulfite suggest that it is a competitive inhibitor with respect to phosphite (Kᵢₛ=16.1±0.1 mM) and an uncompetitive inhibitor with respect to NAD (Kᵢₛ=10.8±0.1 mM) (FIG. 10). Data from fits to the indicated mechanisms and to alternative inhibition mechanisms are presented in the supplementary material for all experiments (Tables 5, 6, 7, and 8).

Mechanism of Action of Phosphite Dehydrogense

Initial studies of PtxD have shown the enzyme to have sequence identity of about 23-34% with the class of D-hydroxy acid dehydrogenases (DHs). Among the conserved residues are three proposed active site residues Arg²³⁷, Glu²⁶⁶, and His²⁹². On the basis of biochemical and crystallographic studies, the roles of these residues in D-hydroxy acid DHs are believed to involve binding of the carboxylate of the substrate by arginine, deprotonation of the substrate alcohol by histidine, and stabilization of the protonated histidine via a catalytic diad with the active site glutamate. However, the transfer of hydride from phosphite to the cofactor may proceed through a variety of mechanistic pathways, including the dissociative, associative, or concerted mechanisms illustrated for the cofactor NAD in EQ. 7.

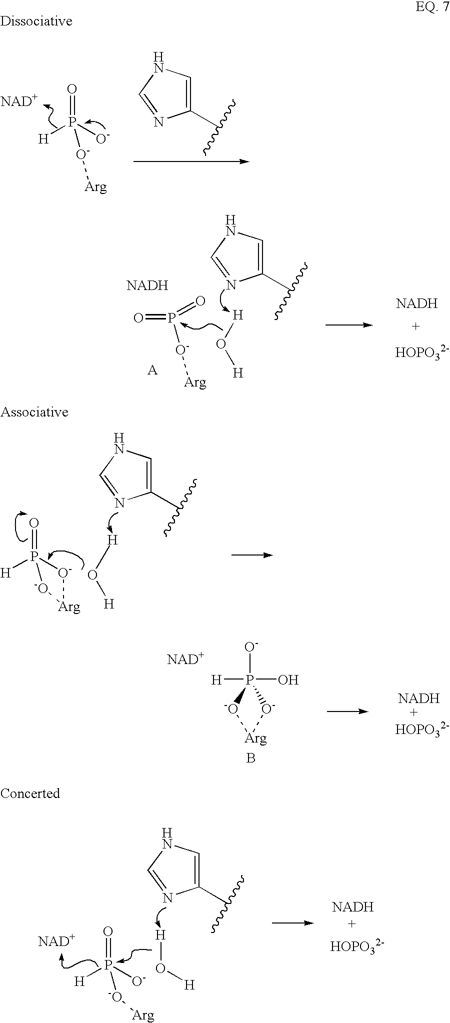

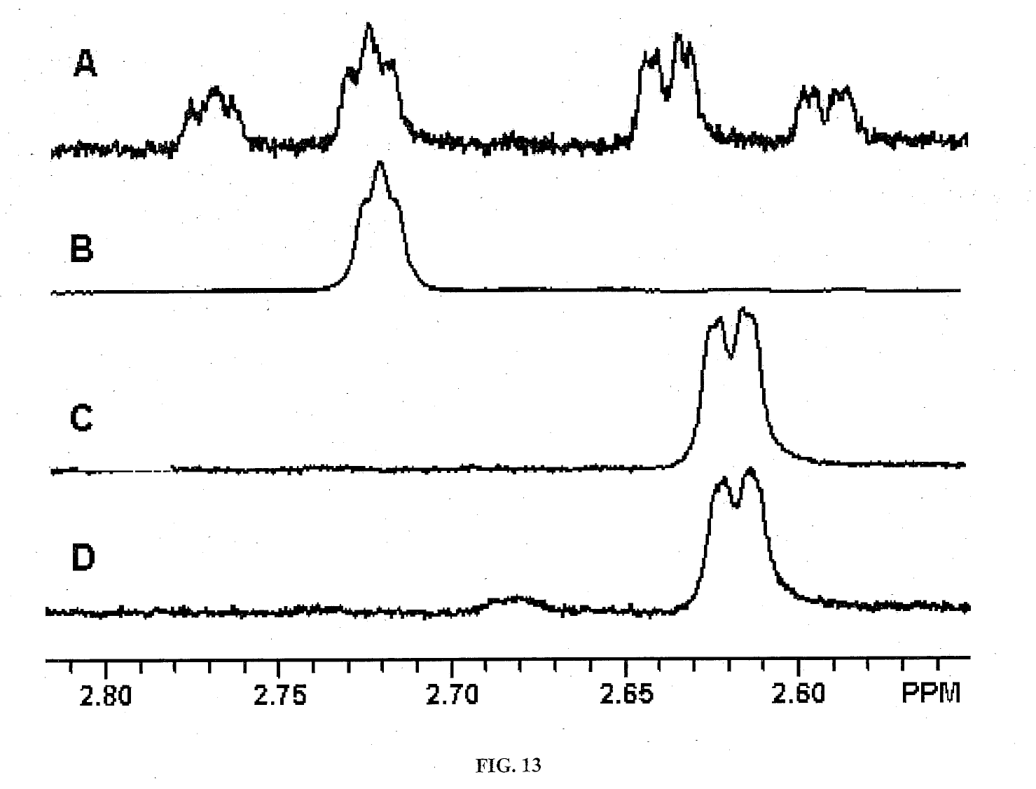

Nevertheless, all of the mechanisms of reduction shown in EQ. 7 involve direct transfer of the phosphorus-bound hydrogen to the cofactor. Consistently, deuterium-labeling experiments demonstrate that the reduction proceeds with direct “hydride” transfer in a stereoselective or stereospecific fashion. Deuterium-labeled phosphite was prepared by repeated lyophilization of phosphorous acid in D₂O. The reaction of the enzyme with this labeled substrate was monitored at 340 nm to ensure complete conversion of the substrate. Given the redox potential at pH 7.0 for NAD⁺/NADH (−0.32V), the equilibrium constant for the oxidation of phosphite by NAD⁺ can be estimated at 10¹¹. The reduced cofactor was purified by anion exchange chromatography, and the ¹H nuclear magnetic resonance (NMR) spectrum was recorded (FIG. 13D) and compared with that of commercial NADH (FIG. 13A). Inspection of the spectral region containing the proton resonances at position 4 of the nicotinamide ring shows that the product is stereoselectively deuterium labeled. Authentic (4R)-[4-²H]-NAD²H (FIG. 13B) and (4S)-[4-²H]-NAD²H (FIG. 13C) we prepared by incubation of glucose dehydrogenase with [1-²H]-glucose and formate dehydrogenase with [1-²H]-formate, respectively, each in the presence of NAD., 13C). By comparison, the NMR spectra show that PtxD transfers a hydride from phosphite to the Re-face of NAD to produce (4S)-[4-²H]-NAD 2H.

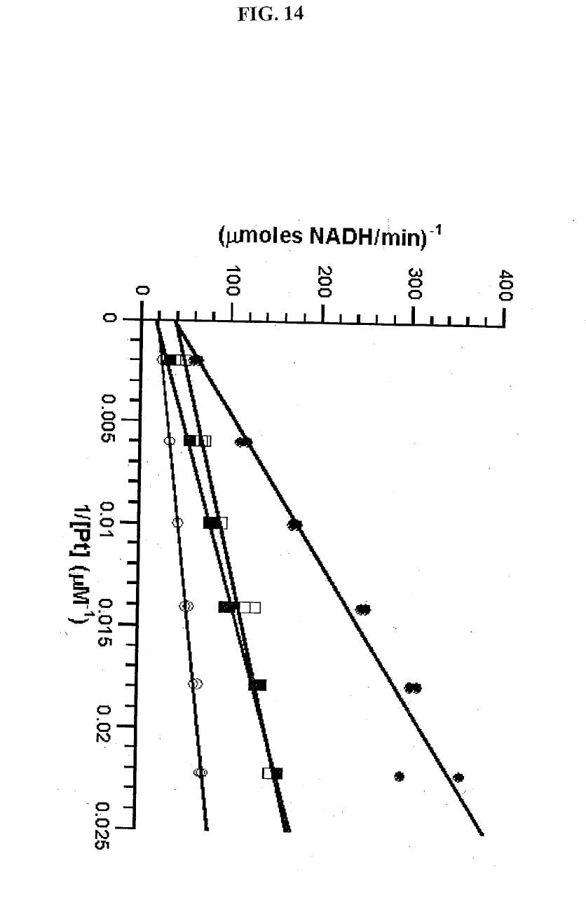

The mechanisms in EQ. 7 differ in the timing of the P—H bond cleavage. For enzymatic reactions, kinetic isotope effects can provide valuable information regarding the relative contribution of the rate constant for a certain chemical step to the overall kinetic process, and/or the extent of X-H bond cleavage in the transition state of this step. Given the support for direct hydride transfer (FIG. 13), the deuterium-labeled phosphite was used to determine whether PtxD displays a kinetic isotope effect on phosphite oxidation. Initial rates were determined at six fixed concentrations of NAD⁺ and six varying concentrations of either labeled or unlabeled phosphite. Control experiments ensured that no exchange occurred between deuterated phosphite and solvent in the time period of the kinetic studies. As shown in FIG. 14 for a subset of these kinetic experiments at two fixed NAD⁺-concentrations, a steady-state kinetic isotope effect of 2.1±0.1 was observed on Vₘₐₓ. Using previously reported stretching frequencies for P—H and P-D bonds in phosphorous acid, the theoretical maximum for a classical kinetic isotope effect for the cleavage of these bonds is estimated to be around 5.0 at 25° C. Therefore, the observed isotope effect on Vₘₐₓ suggests that the hydride transfer step is partially rate limiting, or that deuterium substitution renders this step rate limiting for labeled phosphite. As expected for a steadystate ordered mechanism with the cofactor binding first, essentially no isotope effect (1.0±0.2) was observed on Vₘₐₓ/K_{M, NAD}. The isotope effect on Vₘₐₓ/K_{M, phosphite}, 1.8±0.3, was within experimental error of that for Vₘₐₓ.

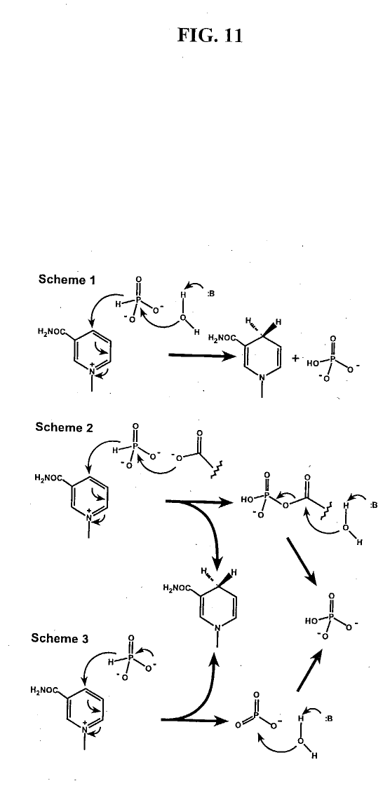

At least two general chemical mechanisms can be envisioned for the conversion redox reaction between phosphite and NAD. The first involves nucleophilic attack at the phosphorus center and subsequent displacement of the hydride to the NAD acceptor. In this mechanism, the nucleophile might arise either from water (FIG. 11, Scheme 1) or from an amino acid side chain on the enzyme (FIG. 11, Scheme 2). In the latter case involving an amino-acid side chain on the enzyme, a phosphoanhydride- or phosphoester-linked enzyme intermediate requiring subsequent hydrolysis would be formed during the reaction. The second mechanism involves initial transfer of the hydride to the NAD acceptor and concomitant formation of the less stable compound metaphosphate (FIG. 11, Scheme 3).

PtxD as a Regenerative Enzyme.

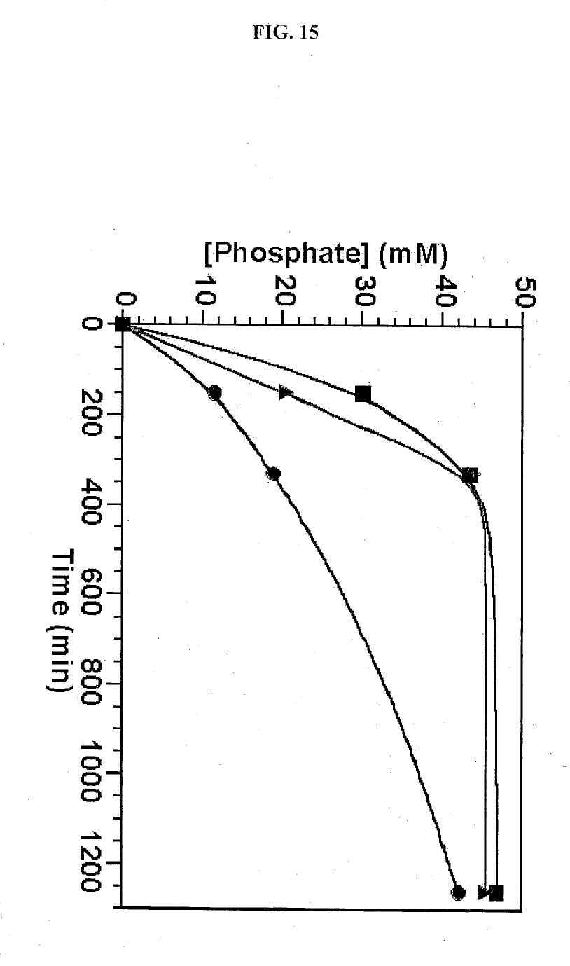

The NADH regeneration reactions using PtxD can be conveniently monitored in three ways. The increase in concentration of the phosphate product can be measured either by a calorimetric assay with a malachite green dye/molybdate complex, or by ³¹P NMR spectroscopy integrating the relative intensities of the resonances of phosphate and phosphite. Alternatively, the synthetic reaction can be monitored by ¹H NMR spectroscopy, integrating the relative intensities of diagnostic peaks for the synthetic substrate and product. Initially, several conditions were assayed for cofactor regeneration varying the amount of cofactor present in the reaction (1:40 or 1:400 NAD⁺:synthetic substrate). The reaction was monitored by the colorimetric method (FIG. 15). It is evident that the reaction reaches completion in either case and that PtxD still remains active after >20 h.

A further reduction of the cofactor concentration resulted in a TTN_{NAD}⁺ of 2000 for both LLDH and HLADH. The TTNs with respect to PtxD were 9.8×10⁴ and 1.6×10⁵ for LLDH and HLADH, respectively, under these conditions (Table 9).

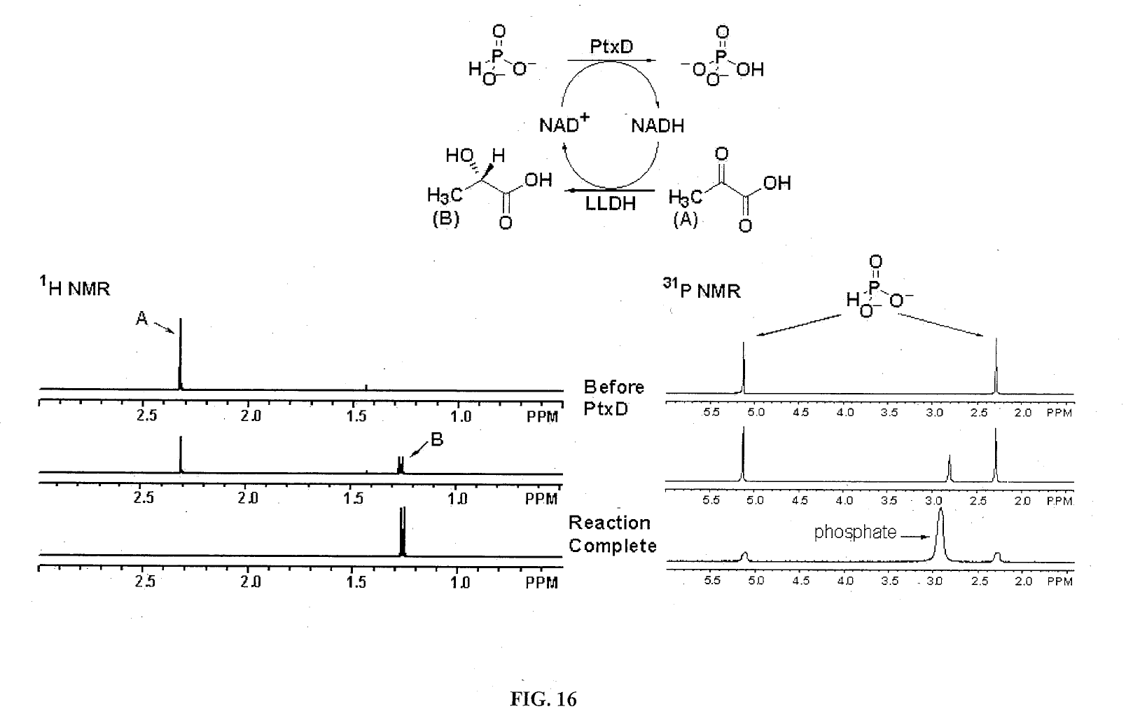

Two examples of reaction progress are shown in FIG. 16 for PtxD with LLDH. The illustrative reaction can be followed by ¹H NMR spectroscopy where the ¹H peak (A) for pyruvate disappears with appearance of peak (B) for L-lactic acid. Alternatively, ³¹P NMR spectroscopy can be used to determine the concentration of phosphate relative to phosphite. Because phosphite is added in excess relative to the synthetic substrate, the phosphorus peak for phosphite does not completely disappear.

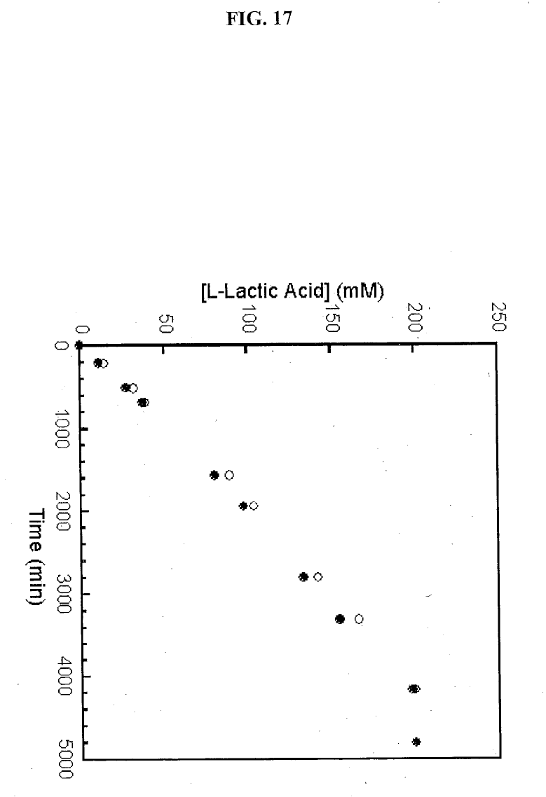

As shown in FIG. 17, a duplicate experiment demonstrated that the velocity of the synthetic system (LLDH) did not decrease over a time span of more than 70 h and that the reaction went to completion. This indicates the high stability and activity of the regenerative enzyme under the reaction conditions. Similar experiments with ADH showed that PtxD can indeed pull the equilibrium of the alcohol dehydrogenase reaction.

Rate-Limiting Step for Cofactor Regeneration.

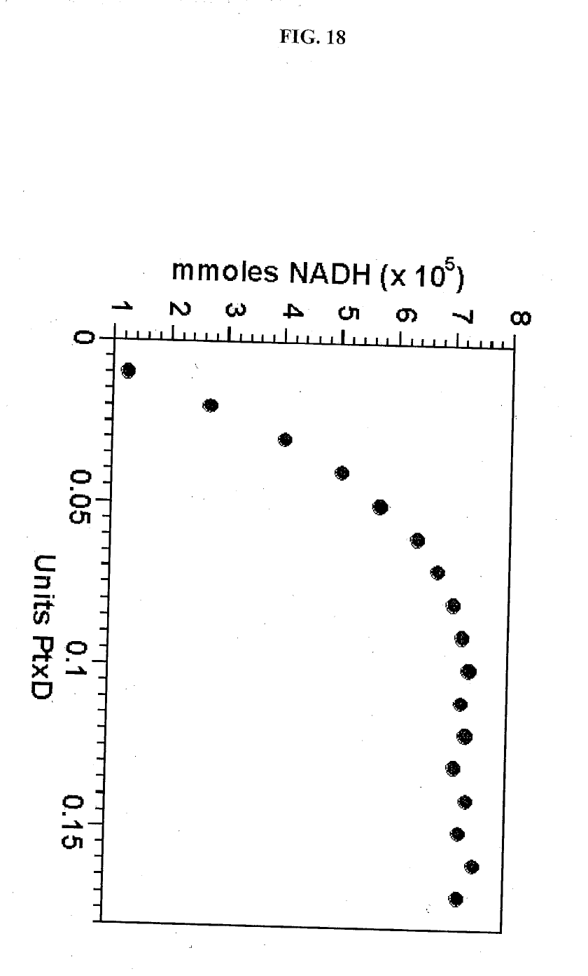

The optimal rates of cofactor regeneration will vary for each synthetic system. The fastest rates will be obtained if the overall process is only limited by the rate constant of the synthetic enzyme. This will be achieved if the cofactor in the reaction is always present as NADH under steady state turnover. In such a scenario, reduction of NAD⁺ to NADH by PtxD is at least 10-fold faster than use of NADH by the synthetic enzyme, and cofactor regeneration is not involved in the rate limiting step of the overall process. As an example of an optimization protocol, a titration experiment was carried out to determine the amount of PtxD needed to render the reaction catalyzed by HLADH completely rate limiting (FIG. 18). A solution containing 0.6 U HLADH, 0.1 U PtxD, 200 mM phosphite, 100 mM acetaldehyde, and 0.1 mM NAD⁺, in 50 mM MOPS buffer, pH 7.25 was monitored for the concentration of NADH after iterative additions of 0.1 U PtxD. The curve levels off at approximately 1.2 units of PtxD or a 2:1 ratio of PtxD:HLADH.

Enzymatic Synthesis of 12-²H]-L-Lactic Acid.

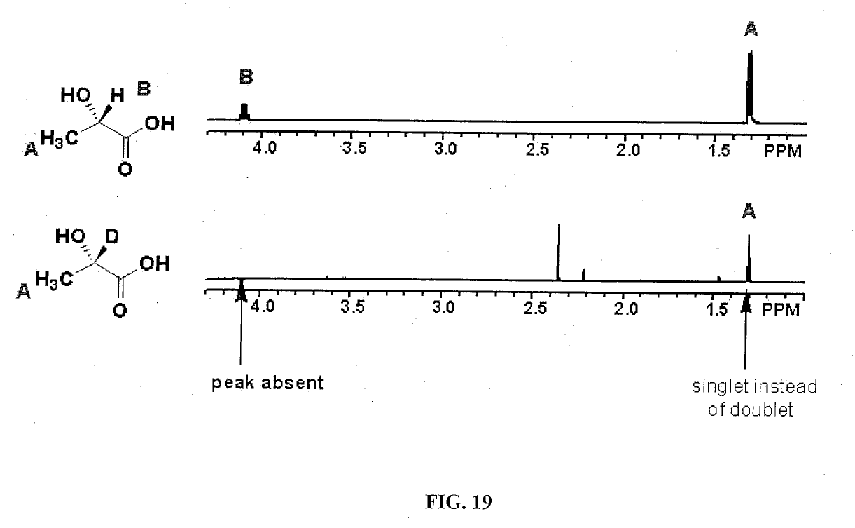

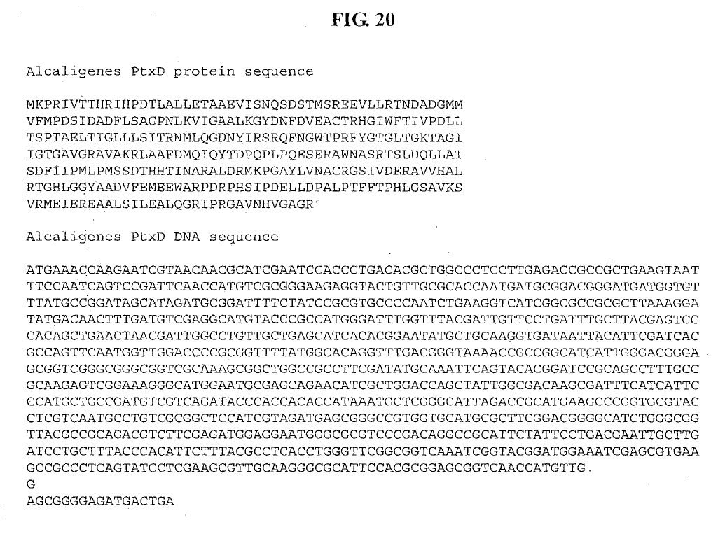

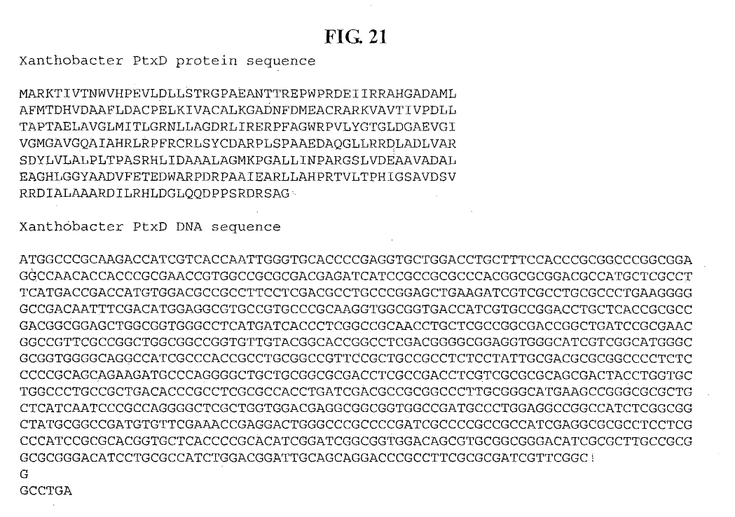

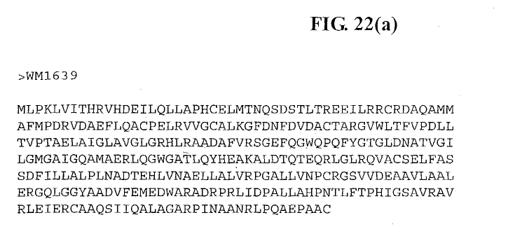

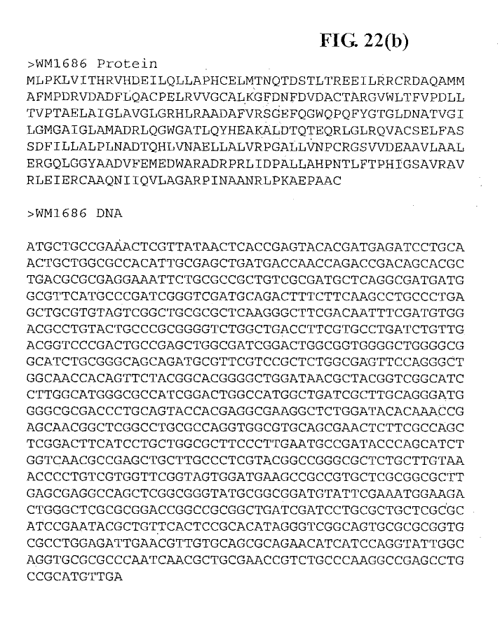

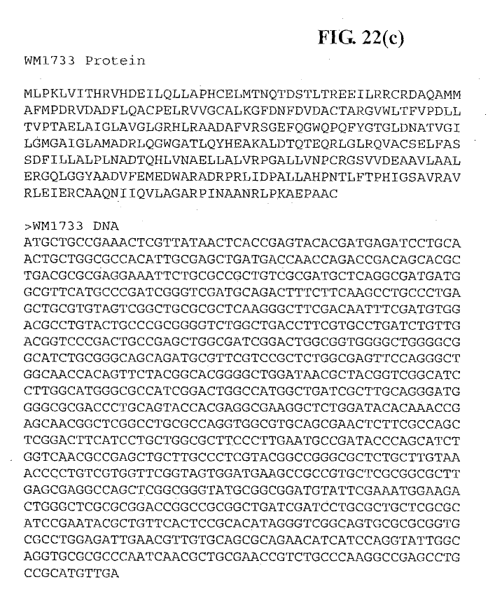

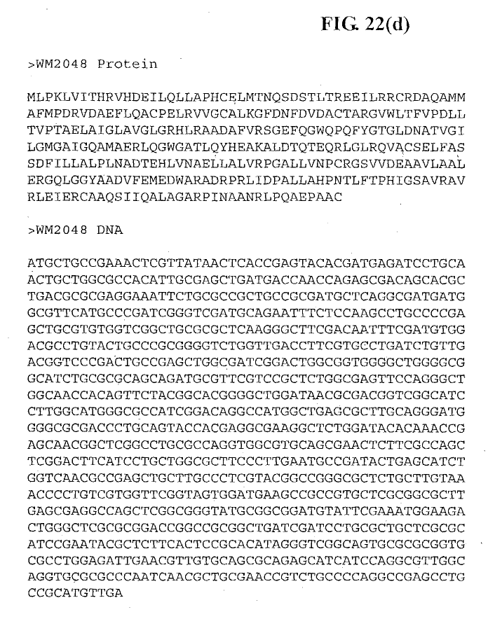

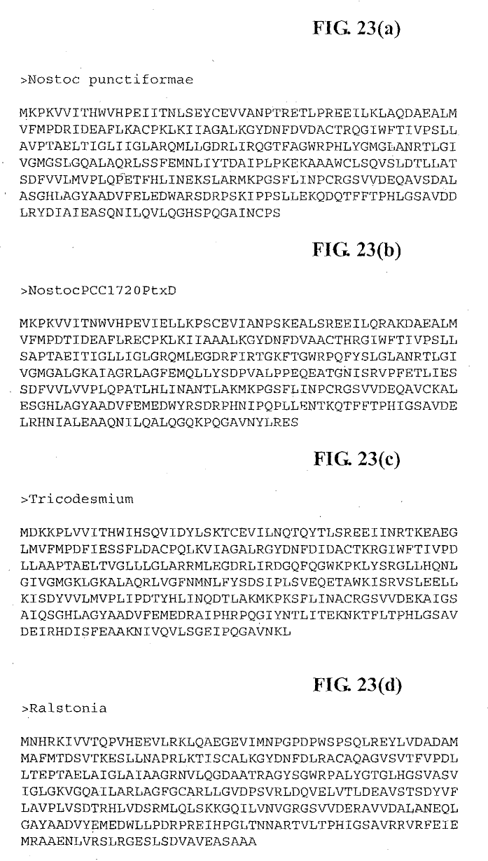

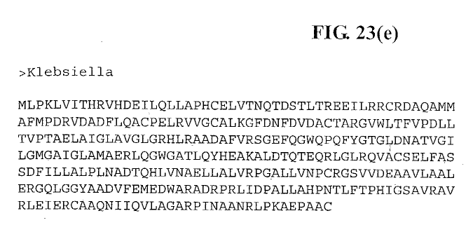

PtxD may be used for the stereoselective incorporation of deuterium into the desired product. PtxD (0.03 mg) was coupled with DLDH (0.05 mg) in a solution containing 20 mM pyruvate, 20 mM deuterium labeled phosphite, and a catalytic amount of NAD⁺ (0.2 mM). As seen in FIG. 19, the ¹H NMR spectrum of unlabeled D-lactate displays a quartet at ˜4.1 ppm associated with the methine hydrogen (—CH(OH)—), and a doublet associated with the terminal methyl hydrogens (CH₃—) at˜1.3 ppm. When pyruvate was reduced to L-lactate by DLDH in the presence of deuterium labeled phosphite and PtxD as regenerative enzyme, the quartet was absent, and the doublet signal associated with the methyl group collapsed to a singlet, as expected upon replacement of the proton at C-2 with a deuterium. The vicinal quadrupole coupling to the deuterium is small, and was not discernable at the resolution used in this experiment. It is appreciated that PtxD may be used as a cofactor regeneration enzyme with D-lactate dehydrogenase as well as other enzymes, as these results demonstrate the viability of PtxD as a tool for stereospecific and cost effective labeling of products.

Materials and Methods

Biological Materials for Regeneration Studies

LLDH (rabbit muscle), DLDH (L. leichmannii), FDH (Candida boidini EC 1.2.1.2), and HLADH (equine liver EC 1.1.1.1) were purchased from Worthington Biochemicals, Roche Molecular Biochemicals, and Sigma-Aldrich. MBP-PtxD was expressed in E. coli and purified using standard affinity methods. All other chemicals were bought from Aldrich, Fisher, or Sigma-Aldrich.

Colorimetric Assay for Determination of Phosphate Concentration

Solutions for the colorimetric assays were prepared as described by Lanzetta and coworkers (Itaya & Ui, 14 Clin. Chim. Acta 361-366 (1966); Lanzetta et al., 100 Anal. Biochem. 95-97 (1979), the disclosures of which are incorporated herein by reference) except that the detergent Sterox was omitted from the dye solution. A 50 μL aliquot of the synthetic reactions were mixed with 800 μL malachite green/ammonium molybdate (MG/AM) solution. After 1 minute, 100 μL of a citrate solution was added to the mixture. After 30 minutes, the absorbance was measured at 660 nm. The concentration of phosphate in the solution was determined from a standard curve prepared independently.

Cofactor Regeneration: PtxD/HLADH and PtxD/LLDH

Solutions for cofactor regeneration contained 0.58 units (4×10⁻⁹ mol) of MBP-PtxD and 0.39 units of LLDH, or 0.44 units (2.5×10⁻⁹ mol) of MBP-PtxD and 0.29 units of LLDH. The reaction mixtures contained 500 mM phosphite, 200 mM pyruvate, 0.1 mM NAD⁺, 10 % D₂O in 50 mM MOPS (morpholinepropane sulfonic acid), pH 7.25 (Vₜₒₜ=2 mL, and 1.5 mL for LLDH and HLADH reactions). Reactions were monitored by ¹H NMR or ³¹P NMR spectroscopy, or the colorimetric dye assay described herein. NMR spectra were taken on Varian 500 MHz or Unity Inova 500NB spectrometers.

Cofactor Regeneration: PtxD/ADH

Solutions for cofactor regeneration contained 0.24 units of MBP-PtxD, 0.12 units of ADH, 50 mM phosphite, 10 mM sodium acetate, 0.1 mM NAD⁺, and 10 mM KCl in 50 mM MOPS, pH 7.25 (Vₜₒₜ=1 mL). Reactions were monitored by ¹H NMR or ³¹P NMR spectroscopy, or the colorimetric dye assay as described herein.

Biosynthesis of [²H]-Lactic Acid

Deuterium labeled lactic acid was prepared in a 5 mL D₂O solution containing 0.05 mg (˜4.4 U) DLDH, 0.03 mg His₆-PtxD, 20 mM pyruvate, 20 mM d-phosphite, and 0.2 mM NAD⁺ in 20 mM NaHCO₃, pD 7.6. The reaction was incubated overnight and monitored by ¹H NMR spectroscopy. The deuterated phosphite was prepared by adding D₂O to phosphorous acid and subsequent lyophilization.

Titration Curve to Determine Optimal Amount of PtxD

The solution contained 200 mM phosphite, 100 mM acetaldehyde, 0.1 U of PtxD, and 0.6 U of HLADH, in 50 mM MOPS, pH 7.25. After blanking the UV-vis reading on this solution, 0.1 mM NAD⁺ (final concentration) was added. PtxD was titrated into the solution and the amount of NADH present at steady state turnover was measured at 260 and 340 nm.

Bacterial Strains and Plasmids

The bacterial strains used in the study are shown in Table 1. In general, DH5a and DH5a/λpir were used as hosts for cloning experiments, while S17-1 and BW20767 were used as donor strains for conjugation experiments involving broad-host-range plasmids. Plasmids pTZ18R, pUC4K, and pSL1180 (5) were obtained from Pharmacia (Piscataway, N.J.). Plasmid pBluescript KS(+) was obtained from Stratagene (La Jolla, Calif.).

Media

Most media used in the study have been previously reported, including Minimal A medium (Miller, in A Short Course in Bacterial Genetics, CSHL Press, Plainview, N.Y., (1992), the disclosure of which is incorporated herein by reference). Antibiotics were used at the following concentrations for E. coli and Pseudomonas stutzeri WM88: kanamycin, 50 μg/ml; ampicillin, 100 μg/ml; tetracycline, 12 μg/ml; and streptomycin, 100 μg/ml. For Pseudomonas aeruginosa, antibiotics were used as follows: carbenicillin (instead of ampicillin), 200 μg/ml; tetracycline, 100 μg/ml; and rifampin, 25 μg/ml. P compounds were prepared fresh and filter sterilized prior to addition to media at a final concentration of 0.5 mM. Noble agar (1.6%) was used to solidify media used for testing P oxidation phenotypes. Sucrose-resistant recombinants of strains carrying the Bacillus subtilis sacB gene as a counterselectable marker were selected on agar-solidified medium containing 10 g of tryptone, 5 g of yeast extract, and 50 g of sucrose per liter. Denitrification was tested in tightly closed screw cap tubes completely filled with Luria-Bertani broth with and without 0.1% NaNO₂ or 0.1% NaNO₃.

P Oxidation Phenotypes

P oxidation phenotypes were scored by growth on 0.4% glucose-MOPS (morpholinepropanesulfonic acid) medium with the compound under study supplied at 0.5 mM as the sole P source. The ability to oxidize a compound to phosphate allows growth on this medium. Because the amount of P required for growth is relatively small, the contaminating levels of phosphate found in many medium components, especially agar, allow slight background growth of all strains in these media. To control for this variable, the strains in question were always compared to suitable positive and negative controls streaked on the same plate.

NMR Spectroscopic Analysis of the P Compounds Used in the Study

The stability of phosphite and hypophosphite in stock solutions and in MOPS medium was analyzed by ³¹P NMR. Spectra were obtained in 10-mm tubes at ambient temperature by using either a General Electric GN500-NB (pulse time, 55 μs; relaxation delay, 3.5 s) or a General Electric GN300-NB (pulse time, 24 μs; relaxation delay, 4 s) instrument. D₂O was added to allow deuterium signal locking to be used. For experiments in which the P concentration ranged from 250 to 1,000 μM, 512 or 1,012 scans were taken for each sample. Fewer scans were used for samples with high P concentrations. No detectable oxidation products of either phosphite or hypophosphite were seen after 2 weeks of incubation under the growth conditions used in this study. Phosphite stock solutions were stable for at least 1 year. However, prolonged storage of hypophosphite stock solutions led to accumulation of phosphite, approaching ca. 50% of the total P after 6 months of storage at 4° C. For this reason, all reduced P stock solutions were prepared fresh as needed, and media containing reduced P compounds were used within 2 weeks of preparation.

DNA Methods

Standard methods were used throughout for isolation and manipulation of plasmid DNA. Chromosomal DNA was isolated from P. stutzeri WM88 by the cetyltrimethylammonium bromide method of Ausubel et al., in Current Protocols in Molecular Biology, John Wiley & Sons, Inc., N.Y. (1992), the disclosure of which is incorporated herein by reference). DNA hybridizations were performed as previously described by Metcalf et al., 180 J. Bacteriol. 5547-5558 (1998), the disclosure of which is incorporated herein by reference). Probes used for hybridization experiments were labeled with [α-³²P]dATP by using the Prime-a-Gene kit (Promega, Madison, Wis.) according to the manufacturer's specifications. DNA sequences were determined from double-stranded templates by automated dye terminator sequencing at the Genetic Engineering Facility, University of Illinois. The initial sequences of each clone were always determined by using standard lacZ forward and reverse primers. The remaining sequences were obtained either with internal primers or from nested deletions constructed with the ExoIII/Mung Bean deletion kit (Stratagene).

Cloning and Analysis of 16S rDNA

16S ribosomal DNA (rDNA) from P. stutzeri WM88 was amplified by PCR from genomic DNA with Vent DNA polymerase (New England Biolabs, Beverly, Mass.) by using the primers 5′-TTGGATCCAGAGTTTGATCMTGGCTCAG-3′ and 5′-GTTGGATCCACGGYTACCTTGTTACGAYT-3′. The PCR products from separate reactions were cloned into pWM73 to generate pWM206 and pWM207. The complete DNA sequences of both clones were determined, and these sequences are in complete agreement. To identify the species, this 16S rDNA sequence was compared to others in the Ribosomal Database Project (http://rdpwww.life uiuc.edu) by utilizing the collection of analysis tools provided at that Internet site.

Plasmid Constructions.

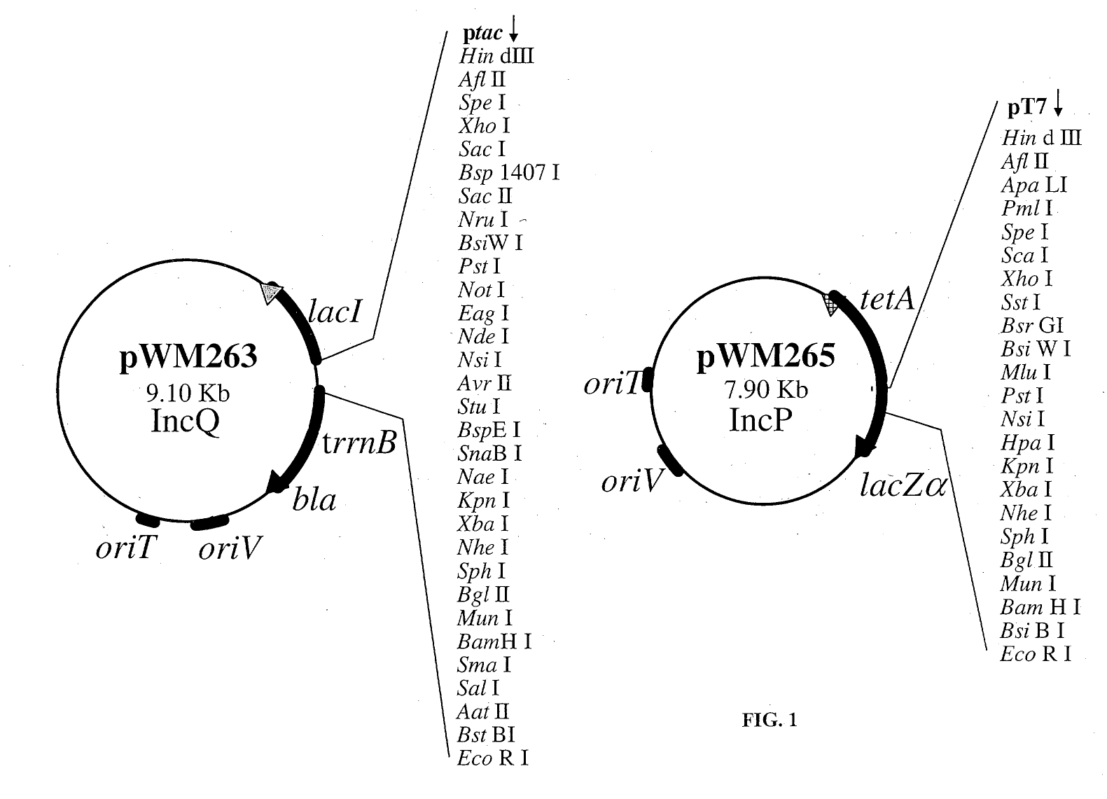

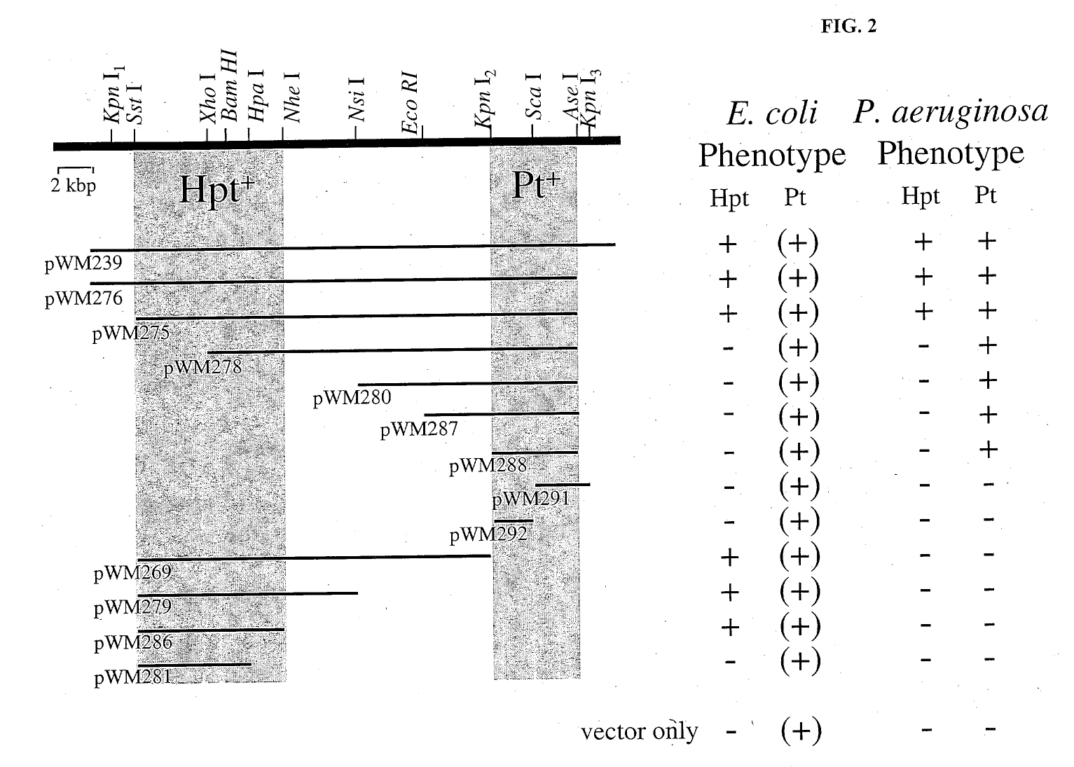

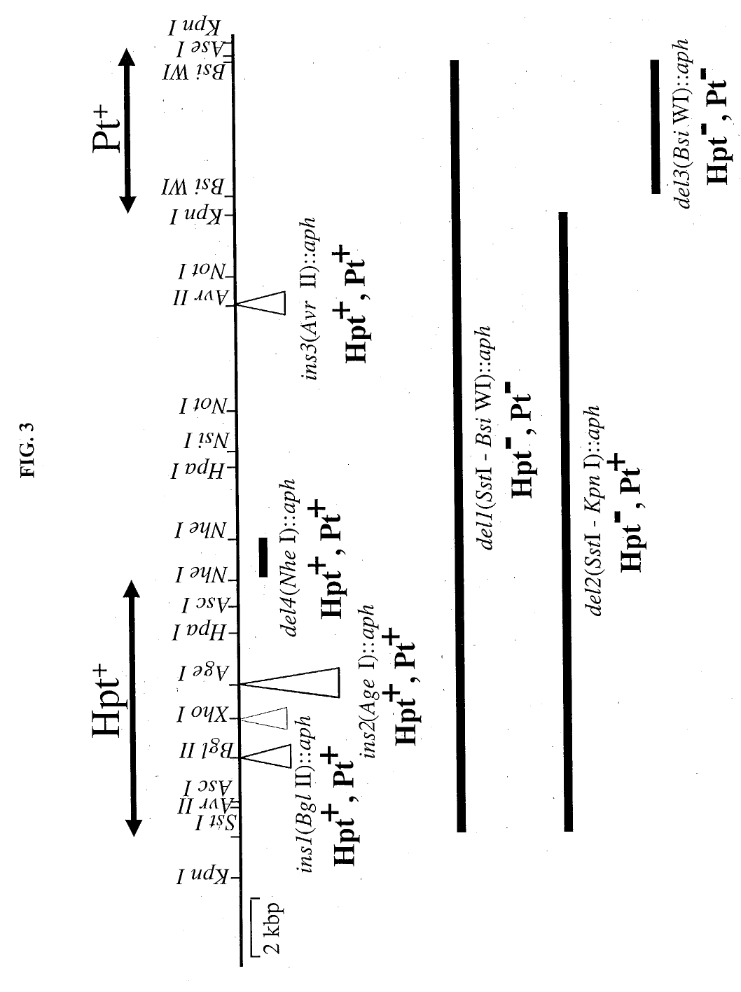

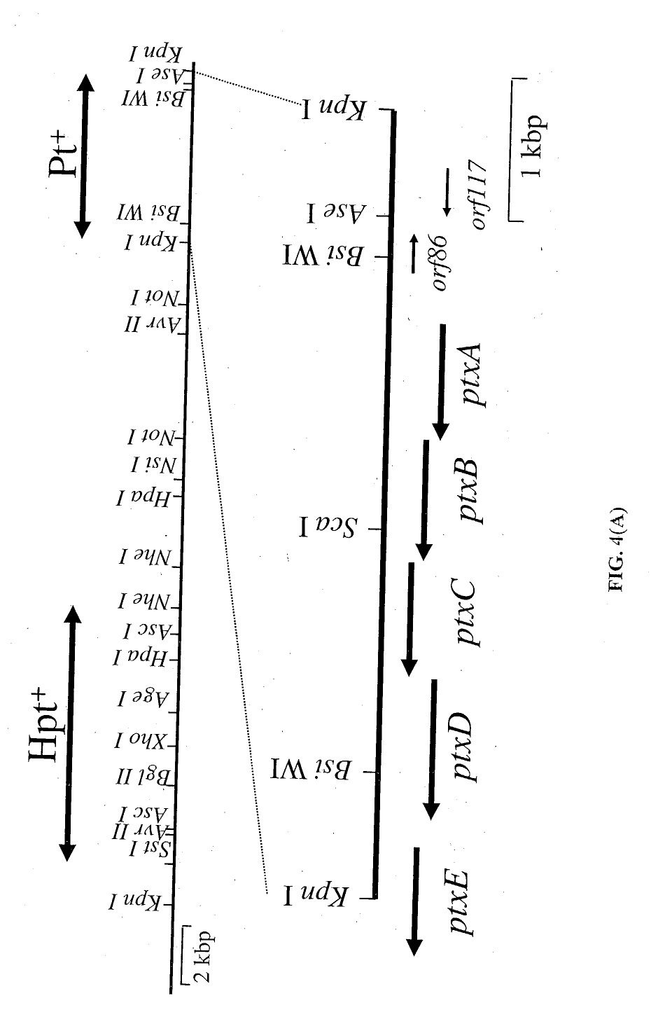

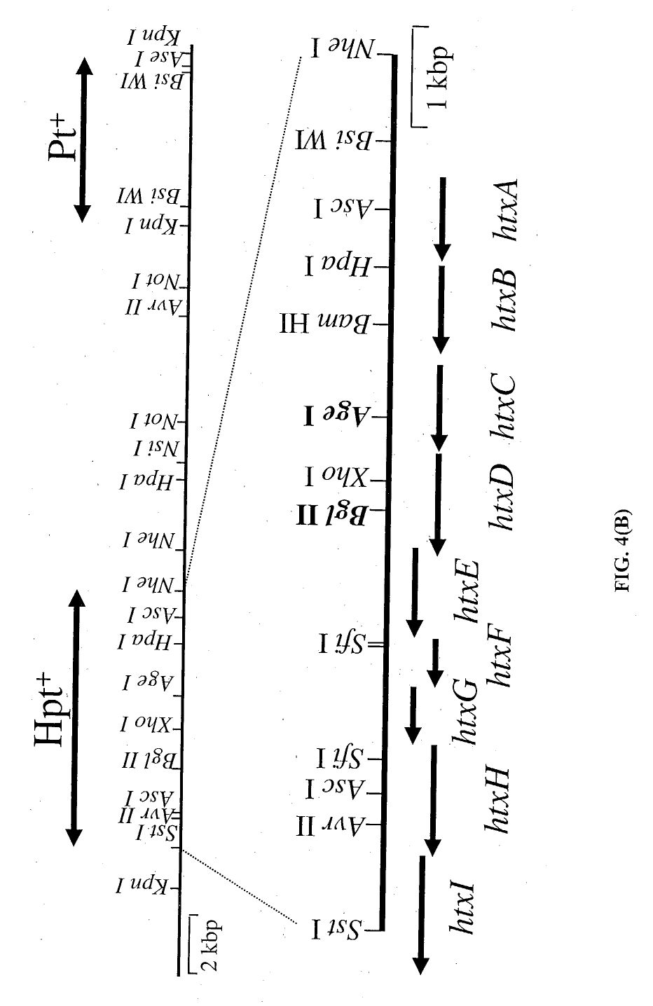

In many cases the restriction sites found within the polylinker of each vector were used for these constructions (FIG. 1). The first set of plasmids was used in subsequent constructions as vectors or as a source for antibiotic resistance cassettes. The broad-host-range IncQ plasmids pWM263 and pWM264 were constructed by replacement of the EcoRI-HindIII polylinkers of pMMB67HE and pMMB67EH, respectively, with the EcoRIHindIII polylinker of pSL1180. Similarly, the broad-host-range IncP plasmids pWM265 and pWM266 were constructed by replacement of the EcoRI-HindIII polylinkers of pDN18 and pDN19, respectively, with the EcoRI-HindIII polylinker of pSL1180. Plasmid pJK25, carrying an aph cassette flanked by symmetrical polylinkers, was constructed by insertion of the 1.3-kbp SalI cassette of pUC4K into the SalI site of pBEND2. Plasmid pJK25 greatly simplifies in vitro construction of gene disruptions by allowing isolation of the aph gene cassette (encoding resistance to kanamycin) by digestion with a single restriction endonuclease, chosen from a variety of different possible enzymes.

A cosmid-based genomic library of P. stutzeri WM88 was constructed by ligation of partially Sau3A-digested chromosomal DNA into BamHI-digested pLAFR5. After in vitro packaging of the cosmid library and transfection into S17-1, clones carrying the plasmids pWM234, pWM235, pWM236, pWM237, pWM238, pWM239, and pWM240 were isolated as ones that grew on glucose-MOPS-hypophosphite medium. Plasmid pWM233 is a randomly chosen clone from this library that was used throughout as a negative control for examining growth of various plasmid-bearing strains on hypophosphite and phosphite media.

A set of plasmids carrying various segments of the cosmid clone pWM239 was used as intermediates for subsequent constructions and for testing P oxidation phenotypes in various hosts. Plasmid pWM262 carries the ca. 23-kbp SstI-to-KpnI fragment of pWM239 cloned into the same sites in pTZ18R, while pWM269 carries the ca. 23-kbp SstI-to-KpnI fragment of pWM262 cloned into the same sites of pWM265. Plasmids pWM273 and pWM274 were constructed by cloning the ca. 30-kbp AseI fragment of pWM239 into the NdeI site of pSL1180; the plasmids differ only in the orientation of the insert. Plasmid pWM275 has the XbaI-to-SstI insert of pWM273 cloned into the same sites in pWM265. Plasmid pWM276 has the XbaI-to-MluI insert of pWM273 cloned into the same sites in pWM265. Plasmid pWM277 has the XbaI-to-MluI insert of pWM274 cloned into the same sites in pWM265. Plasmids pWM284 and pWM285 have the 5.8-kbp KpnI fragment of pWM239 cloned into the same site of pWM265 in opposite orientations. A series of deletion derivatives of various plasmids were constructed that removed all DNA between a polylinker restriction site and the most distal site within the inserted region for the same enzyme. These were pWM278 (pWM276 D XhoI), pWM279 (pWM275 D NsiI), pWM280 (pWM277 D NsiI), pWM281 (pWM275 D HpaI), pWM282 (pWM279 D BamHI), pWM286(pWM279 D NheI), pWM287 (pWM280 D EcoRI), pWM288 (pWM277 D KpnI), pWM291 (pWM284 D ScaI), and pWM292 (pWM285 D ScaI).

Another set of plasmids was used for the construction of deletion and insertion mutations in P. stutzeri WM88 as described herein. Plasmid pWM296 has the ca. 5.9-kbp XbaI-to-SmaI fragment of pWM284 cloned into SpeI- and SmaI-digested pWM95. Plasmid pWM304 has the ca. 6-kbp AscI fragment of pWM275, made blunt by treatment with deoxynucleoside triphosphates (dNTPs) and T4 DNA polymerase, cloned into the SmaI site of pWM95. Plasmid pWM305 has the ca. 6-kbp HpaI fragment of pWM275 cloned into the SmaI site of pWM95. Plasmid pWM306 has the ca. 4.5-kbp NotI fragment of pWM275 cloned into the NotI site of pWM95. Plasmid pWM298 was constructed by insertion of the PstI-aph cassette of pUC4K into BsiWI-digested pWM296 after treatment of both vector and insert with dNTPs and T4 DNA polymerase. Plasmid pWM322 was constructed by insertion of the XmaI-aph cassette of pJK25 into the AgeI site of pWM304. Plasmid pWM323 was constructed by insertion of the BamHI-aph cassette of pJK25 into the BglII site of pWM304. Plasmid pWM324 was constructed by insertion of the NheI-aph cassette of pJK25 into pWM305 with its 1.2-kbp NheI fragment deleted. Plasmid pWM326 was constructed by insertion of the NheI-aph cassette of pJK25 into the AvrII site of pWM306. Plasmid pWM260 has the DraI-to-NsiI fragment of pWM239 cloned into PstI- and SmaI-cut pBluescript KS(1). Plasmid pWM261 has the DraI-to-NsiI fragment of pWM238 cloned into PstI- and SmaI-cut pBluescript KS(1). Plasmid pWM338 was constructed by cloning the ca. 1.3-kbp SstI fragment of pWM260 into the SstI site of pWM284. Plasmid pWM340 was constructed by cloning the ca. 5.0-kbp SstI fragment of pWM261 into the SstI site of pWM284. Plasmid pWM342 was constructed by insertion of the EcoRV-aph cassette of pJK25 into pWM338 with an internal ca. 5.0-kbp BsiWI fragment deleted after treatment with dNTPs and T4 DNA polymerase. Plasmid pWM344 was constructed by insertion of the MluI-aph cassette of pJK25 into the Mlul site of pWM340. Plasmid pWM346 was constructed by insertion of the ApaI-to-PmlI fragment of pWM342 into Apaland SmaI-cut pWM95. Plasmid pWM347 was constructed by insertion of the ApaI-to-PmlI fragment of pWM344 into ApaI- and SmaI-cut pWM95.

The plasmids used for sequence determinations were pWM294 and pWM360. Plasmid pWM294 carries the 5.8-kbp KpnI fragment of pWM239 cloned into the KpnI site of pBluescript KS(1). Plasmid pWM360 was constructed by digestion of pWM262 with XbaI and NheI and subsequent ligation of the compatible XbaI and NheI ends.

Genetic Techniques

In general, conjugation between E. coli donors and P. aeruginosa or P. stutzeri recipients was performed by mixing donor and recipient cells in a 10:1 ratio and incubating overnight on TYE agar. Cells from the mating mixture were then scraped from the surface and resuspended in basal medium, and various aliquots were spread onto selective agar. The genomic library of P. stutzeri WM88 in pLAFR5 was moved into P. aeruginosa PAK en masse by replica plating master plates of the library in E. coli S17-1 onto a lawn of P. aeruginosa PAK. After overnight incubation, these plates were replica plated onto minimal A medium-tetracycline agar to select for exconjugates. In general, the P oxidation phenotypes of various plasmid subclones in P. aeruginosa were examined in strain P. aeruginosa PAK Dpil rif. Plasmids were moved into this strain by conjugation with E. coli BW20767 or S17-1 donors with selection on TYE agar with rifampin in combination with either tetracycline or carbenicillin, as appropriate. Exconjugants of E. coli donors and P. stutzeri WM567 recipients were selected on glucose-MOPS medium with an appropriate antibiotic. Exconjugates of E. coli donors and either WM581, WM688, or WM691 were selected on TYE agar with kanamycin and tetracycline.

In vitro-constructed mutations of cloned genes were recombined onto the P. stutzeri WM567 chromosome. To do this, various segments of the original cosmid clones pWM238 and pWM239 were subcloned into pWM95. Plasmid pWM95 is a suicide vector that can be transferred to a wide variety of gram-negative organisms by conjugation and carries a counterselectable sacB marker. In vitro deletion and insertion mutations carrying a selectable marker for kanamycin resistance, aph, were made in these clones and recombined onto the chromosome in a two-step process. In the first step, the plasmids carrying the mutations were integrated into the P. stutzeri WM567 chromosome by selection for kanamycin- and streptomycin-resistant exconjugates after mating with E. coli BW20767 donors. In the second step, recombinants that had lost the plasmid backbone were obtained by selection against the plasmid-carried sacB gene by sucrose resistance. Finally, these recombinants were screened for the presence of the desired mutation by scoring kanamycin resistance. The mutant strains reported here and plasmids used for their construction were as follows: P. stutzeri WM581 from pWM298, P. stutzeri WM678 from pWM322, P. stutzeri WM679 from pWM323, P. stutzeri WW680 from pWM324, P. stutzeri WM682 from pWM326, P. stutzeri WM688 from pWM346, and P. stutzeri WM691 from pWM347. Each mutant was verified to have the predicted structure by hybridization of restriction endonuclease-digested genomic DNAs to labeled pJK25 and pWM273, after agarose gel electrophoresis and blotting to positively charged nylon membranes.

Nucleotide Sequence Accession Numbers

The GenBank accession numbers for the P. stutzeri WM88 DNA sequences determined in this study are AF038653 for 16S rDNA, AF061070 for the minimal region required for the oxidation of phosphite to phosphate, and AF061267 for the minimal region required for oxidation of hypophosphite to phosphite.

Organisms and Culture Conditions

E. coli DH5a (Grant et al., 87 Proc. Natl. Acad. Sci. 4645-4649 (1990), the disclosure of which is incorporated herein by reference) was used as the host for DNA cloning experiments, and E. coli BL21(DE3) (Studier et al., 185 Methods Enzymol. 60-89 (1990), the disclosure of which is incorporated herein by reference) was used as the host for overexpression from plasmid pET11a (Novagen, Inc., Madison, Wis.) and its derivatives. These strains were grown in standard LB medium supplemented with ampicillin (50 μg/ml) or carbenicillin (100 μg/ml) as needed. All P. stutzeri strains are derivatives of the phosphite- and hypophosphite-oxidizing bacterium P. stutzeri WM88. P. stutzeri WM536 is a mutant that does not produce extracellular capsule. P. stutzeri WM567 is a streptomycin-resistant derivative of P. stutzeri WM536. P. stutzeri WM581 (rpsL, del3(BsiWI)::aph) is a derivative of P. stutzeri WM567 that carries a deletion of the ptxABCDE operon and is unable to utilize either phosphite or hypophosphite as sole phosphorus sources. P. stutzeri strains were grown at 37° C. in 0.4% glucose-MOPS¹ medium containing the indicated phosphorus source at 0.5 mM unless otherwise noted. Phosphite and hypophosphite were always prepared fresh and filtersterilized prior to use. Cells were grown in 0.4% glucose-MOPS medium with 0.1 mM phosphate for studies involving phosphate-limited growth. Cells were grown in 0.12% glucose-MOPS medium with 2.0 mM phosphate for studies involving phosphate-excess growth. For large scale protein purifications, P. stutzeri WM536 was grown in a 30-liter stainless steel bioreactor (model P30A, B. Braun Biotech, Allentown, Pa.) at 30° C. in glucose-MOPS medium containing 2 mM hypophosphite. Antifoam 289 (Sigma) was added as needed. To ensure that no residual phosphate was present in the media, all glassware was soaked and rinsed with ultrapure deionized water prior to use. The bioreactor was rinsed with 0.1 M nitric acid prior to use for the same purpose.

Cloning and Overexpression of ptxD

Standard methods for isolation and manipulation of plasmid DNA were used throughout (Ausubel et al., in Current Protocols in Molecular Biology, John Wiley & Sons, Inc., N.Y. (1992), the disclosure of which is incorporated herein by reference). The ptxD gene was amplified by polymerase chain reaction from plasmid pWM294 using Vent DNA polymerase (Life Technologies, Inc.) and the primers 5′-CACACACATATGCTGCCGAAACTCG-3′ and 5′-AGCGGATAACAATTTACAGG-3′. The forward primer was designed to introduce an NdeI site (underlined) at the ptxD initiation codon. The resulting polymerase chain reaction product was digested with NdeI and BamHI and cloned into the same sites in the expression vector pET11a (Novagen, Inc., Madison, Wis.) to form pWM302. The ptxD gene in pWM302 was sequenced with standard T7 promoter and terminator primers at the W. M. Keck Center for Comparative and Functional Genomics (University of Illinois).

To induce overexpression of plasmid-borne genes, E. coli BL21 (DE3) transformants carrying either pWM302 or pET11a were grown in LB medium containing carbenicillin at 37° C. Upon reaching midlog phase (A₆₀₀˜0.6), IPTG (1 mM final concentration) was added, and the cultures were incubated for an additional 1.5 h, at which time they were harvested by centrifugation. For large scale overexpression experiments, cultures were grown in the 30-liter stainless steel bioreactor at 30° C.

Purification Steps

All purification steps took place at 4° C. Approximately 20 g (wet weight) of IPTG-induced BL21 (DE3)/pWM302 cells were resuspended in 35 ml of freshly made buffer A (20 mM MOPS buffer, pH 7.25, 10% glycerol, 1 mM dithiothreitol). DNase I (˜10 mg) was added, and the suspension was passed twice through a chilled French pressure cell at 13,000 p.s.i. The broken cell slurry was then centrifuged at 20,000×g for 30 min to pellet debris and unbroken cells, and the supernatant fraction was collected as the crude cell extract. The crude extract was separated into soluble and membrane fractions by centrifugation at 270,000×g for 45 min. The pellet was discarded, and the supernatant fraction (high speed extract) was used in subsequent steps.

High speed extracts containing ˜180-350 mg of protein were loaded onto an NAD-affinity column (˜10 ml of swollen resin) with attachment of the ligand at C-8 (catalog no. N1008; Sigma) at a flow rate of 0.5 ml/min. Fractions from the flow-through containing PtxD activity were pooled, adjusted to 1 M NaCl, and loaded at the same flow rate onto an NAD affinity column (˜15 ml of swollen resin) with attachment of the ligand at N-6 (catalog no. N9505; Sigma). Unbound proteins were eluted from the second column with 10 column volumes of buffer B (20 mM MOPS, pH 7.25, 10% glycerol, 1 mM dithiothreitol, 1M NaCl) followed by 10 column volumes of buffer A. PtxD was then eluted with an NAD gradient (0-3 mM) in buffer A over 5 column volumes. Active fractions that were homogenous as determined by visual inspection of SDS-PAGE gels were pooled and then desalted and concentrated by ultrafiltration (Centriplus membrane; molecular mass cut-off 30,000 Da; Amicon, Beverly, Mass.).

PtxD from P. stutzeri WM536 was purified following the same tandem affinity protocol. Eluted fractions with specific activity higher than about 3.0 units/mg were pooled and purified through the tandem affinity protocol a second time. Active fractions from the second purification that were ˜90% pure as determined by visual inspection of SDS-PAGE gels were pooled and concentrated as described herein.

Protein and Enzyme Assays

PtxD activity was assayed spectrophotometrically by continuously monitoring the absorbance of NADH at 340 nm. The extinction coefficient of 6220 M⁻¹ cm⁻¹ was used to calculate the concentration of NADH. Standard enzyme units (μmol of NADH produced min⁻¹) are used throughout. Unless otherwise noted, the assay mixture contained 20 mM MOPS, pH 7.25, 0.5 mM NAD, 1 mM phosphite, and 10-100 μl of enzyme extract in a 1-ml volume. Most assays were carried out at room temperature. Characterization assays were carried out at 30° C. For the temperature studies, acetylated bovine serum albumin (10 μg/ml final concentration) was added to the assay buffer. For the pH studies, the MOPS buffer was replaced by a Tris/acetate/MES buffer (100 mM Tris, 50 mM glacial acetic acid, and 50 mM MES), and the pH was adjusted with HCl or NaOH. The ionic strength of this buffer was calculated to be 0.1 at all pH values. Phosphate production was assayed colorimetrically by end point assays (Lanzetta et al., 100 Anal. Biochem. 95-97 (1979), the disclosure of which is incorporated herein by reference) Protein concentrations were assayed with Coomassie Plus reagent from Pierce according to manufacturer protocols with bovine serum albumin as the standard.

Gel Electrophoresis

SDS-PAGE was carried out as described by Laemmli (227 Nature 680-685 (1970), the disclosure of which is incorporated herein by reference) in 12% polyacrylamide slab gels. Proteins were visualized by staining with Coomassie Blue. Native PAGE was carried out at 4° C. in 6% polyacrylamide continuous gels using a 35 mM HEPES, 43 mM imidazole buffer (final pH 7.1). Two activity stains were used. To detect phosphite-dependent NADH production, native PAGE gel slabs were incubated for 30 min at 30° C. in 100 ml of 100 mM Tris, pH 8.5, containing 10 mM phosphite, 25 mg of NAD, 30 mg of nitro blue tetrazolium, and 2 mg of phenazine methanosulfate as described by Heeb & Gabriel (104 Methods Enzymol. 416-439 (1984), the disclosure of which is incorporated herein by reference). Chemical reduction of the nitro blue tetrazolium dye by enzymatically produced NADH results in precipitation of a dark blue product, which is easily seen in the stained gels. To detect phosphate production from phosphite and NAD, native PAGE gel slabs were incubated in 100 ml of 100 mM Tris, pH 8.5, containing 10 mM phosphite, 25 mg of NAD, and 50 mM calcium chloride. The gels were then rinsed and stained with ammonium molybdate and methyl green as described by Cutting (104 Methods Enzymol. 451-455 (1984), the disclosure of which is incorporated herein by reference). Phosphate produced by the enzymatic oxidation of phosphite is precipitated as CaHPO₄, which is visualized as a dark green band by the staining procedure.

The abbreviations used are: MOPS, 3-N-morpholinopropanesulfonic acid; PAGE, polyacrylamide gel electrophoresis; MES, 4-morpholineethanesulfonic acid; IPTG, isopropyl-1-thio-β-D-galactopyranoside.

Gel Filtration and Mass Spectrometry

Gel filtration was carried out in a XK 16/70 column (Amersham Pharmacia Biotech) with Sephacryl S-300 as the matrix. The mobile phase was buffer A with 0.5M NaCl, and the flow rate was 0.5 ml/min. A mixture of purified PtxD and the following standards was applied to the column for estimation of the native molecular mass of PtxD: bovine thyroglobulin (670,000 Da), bovine γ-globulin (158,000 Da), chicken ovalbumin (44,000 Da), horse myoglobin (17,000 Da), and vitamin B₁₂ (1350 Da). Mass spectrometry was carried out at the University of Illinois Mass Spectrometry facility using matrix-assisted laser desorption ionization in a Voyager-DE STR mass spectrometer (PerSeptive Biosystems, Framinghan, Mass.).

Amino Terminus Sequencing

Purified PtxD was separated by electrophoresis under denaturing conditions in 12.5% polyacrylamide gels. The protein was then transferred onto a polyvinylidene difluoride membrane (Bio-Rad) using a Hoeffer Scientific semidry blotter according to manufacturer protocols and using Tris-glycine/methanol/SDS as the blotting buffer. Protein was visualized with Coomassie Blue and sequenced by Edman degradation at the University of Illinois Protein Sciences Facility.

TABLE 1
Representative Purifications of PtxD from E. coli
BL21(DE3)/pWM302 and from P. stutzeri WM536.

| | | | Specific | | |
|---|---|---|---|---|---|
| | Activity | Protein | Activity | Yield | Purfication |
| | Units | mg | Units/mg | % | fold |
| E. coli | | | | | |
| BL21 (DE3)/ | | | | | |
| pWM302 | | | | | |
| High speed | 61.4 | 288 | 0.21 | 100.0 | 1.0 |
| supernatant | | | | | |
| NAD affinity (C-8) | 56.7 | 236 | 0.24 | 92.3 | 1.1 |
| NAD affinity (N-6) | 30.3 | 4.65 | 6.52 | 49.3 | 31.0 |
| P. stutzeri WM536 | | | | | |
| High speed | 66.4 | 5553 | 0.01 | 100.0 | 1.0 |
| supernatant | | | | | |
| NAD affinity (C-8) | 62.7 | 4185 | 0.02 | 94.4 | 2.0 |
| NAP affinity (N-6) | 11.9 | 2.28 | 5.20 | 17.9 | 520.0 |
| Second round | 9.4 | 1.81 | 5.20 | 17.9 | 520.0 |
| NAD affinity (C-8) | 9.0 | 1.64 | 5.47 | 13.6 | 547.0 |
| NAD affinity (N-6) | 6.2 | 0.99 | 6.27 | 9.3 | 627.0 |

TABLE 2
Substrate specificity and inhibition of PtxD.

| | Activity | | Activity |
|---|---|---|---|
| Cofactors^{a} | % | Substrates^{b} | % |
| NAD, 1 mM | 100.00 | Phosphite | 100.00 |
| NADP, 1 mM | 0.4 | Nitirite | ND^{c} |
| NADP, 2 mM | 1.1 | Formate | ND |
| NADP, 4 mM | 3.4 | D-Glycerate | ND |
| NADP, 6 mM | 7.1 | D-2-Hydroxy-4-methylvalerate | ND |
| | | D-3-Phosphoglycerate | ND |
| | | DL-Hydroxyisocaproate | ND |
| | | Methylphosphonate | ND |
| | | Aminoethylphosphonate | ND |
| | | Arsenite | ND |
| | | DL-Lactate | ND |
| | | Hypophosphite | ND |
| | | Sulfite | ND |
| Cofactor analog | Activity | Substrate analog | Activity |
| inhibitors^{d} | % | inhibitors^{e} | % |
| None | 100.00 | None | 100.00 |
| NADH | ND | Phosphate | 116.2 |
| NADP | 78.7 | Nitrite | 77.8 |
| AMP | 105.3 | Formate | 87.6 |
| ADP | 72.8 | D-Glycerate | 62.2 |
| ATP | 77.1 | D-2-Hydroxy-4-methylvalerate | 42.3 |
| ADPR | 80.4 | D-3-Phosphoglycerate | 109.4 |
| | | DL-Hydroxyisocaproate | 88.8 |
| | | Methylphosphonate | 110.4 |
| | | Aminoethylphosphonate | 107.5 |
| | | Arsenite | 39.6 |
| | | DL-Lactate | 122.8 |
| | | Hypophosphite | 96.4 |
| | | Sulfite | ND |

^{a}Cofactors were added at the indicated concentrations with phosphite held at 1.0 mM.
^{b}Substrates were added at 4 mM with NAD held at 1.0 mM.
^{c}ND not detected; the minimum level of detection is ˜0.01 units/mg.
^{d}Cofactor analogs were added at 4 mM concentration in addition to 50 μm NAD and 1.0 mM phosphite.
^{e}Substrate analogs were added at 4 mM concentration in addition to 50 μM phosphite and 1.0 mM NAD.

TABLE 3
Initial rate model fitting for PtxD^{a}.

| Mechanism | Kₐ | K_{b} | Vₘₐₓ | Sigma | variance |
|---|---|---|---|---|---|
| Sequential | 53.1 ± | 54.6 ± | 12.2 ± | 0.0526 | 0.00276 |
| | 6.66 | 69 | 0.226 | | |
| Equilibrium | 4200 ± | 19.8 ± | 10.5 ± | 0.335 | 0.112 |
| ordered | 1760 | 14.0 | 0.513 | | |
| Ping pong | 12700 ± | 12800 ± | 1170 ± | 4.29 | 18.4 |
| | 25600 | 25900 | 4250 | | |

^{a}The data presented in FIG. 8, left panel (phosphite as “A”, NAD as “B”) were analyzed using the program KinetAsyst, which uses an adaptation of the algorithm of Cleland. The data were fit to the indicated enzymatic mechanisms and the values of the key parameters are shown. Concentrations are in μM

TABLE 4
Initial rate model fitting for PtXD^{a}.

| Mechanism | Kₐ | K_{b} | Vₘₐₓ | Sigma | Variance |
|---|---|---|---|---|---|
| Sequential | 54.6 ± | 53.1 ± | 12.2 ± | 0.0526 | 0.00276 |
| | 6.69 | 6.65 | 0.226 | | |
| Equilibrium | 4520 ± | 17.7 ± | 10.4 ± | 0.379 | 0.143 |
| ordered | 2250 | 15.2 | 0.563 | | |
| Pingpong | 12800 ± | 12700 ± | 1170 ± | 4.29 | 18.4 |
| | 25900 | 25600 | 4250 | | |

^{a}The data presented in FIG. 8, right panel (NAP as “A”, phosphite as “B”) were analyzed using the program KinetAsyst, which uses an adaptation of the algorithm of Cleland. The data were fit to the indicated enzymatic mechanisms and the values of the key parameters are shown concentrations are in μM.

TABLE 5
Inhibition of PtxD by NADH with NAD constant at 50 μM^{a}.

| Mechanism | Vₘₐₓ | K_{M} | Kᵢₛ | Kᵢᵢ | Sigma | Variance |
|---|---|---|---|---|---|---|
| Competitive | 4.62 ± | 1050 ± | 116 ± | NA | 0.0351 | 0.00123 |
| | 0.211 | 72.6 | 6.43 | | | |
| Non- | 4.49 ± | 1020 ± | 110 ± | −1550 ± | 0.0355 | 0.00126 |
| competitive | 0.270 | 92.3 | 13.3 | 3500 | | |
| Un-competitive | 9.48 ± | 2850 ± | NA | 35.2 ± | 0.0735 | 0.00540 |
| | 2.00 | 721 | | 9.18 | | |

^{a}The data presented in FIG. 9, left panel, were analyzed using the program KinetAsyst, which uses an adaptation of the algorithm of Cleland. The data were fit to the indicated inhibition mechanisms and the values of the key parameters are shown. Concentrations are in μM.
NA indicates not applicable.

TABLE 6
Inhibition of PtxD by NADH with Phosphite constant at 50 μM^{a}.

| Mechanism | Vₘₐₓ | K_{M} | Kᵢₛ | Kᵢᵢ | Sigma | Variance |
|---|---|---|---|---|---|---|
| Competitive | 4.81 ± | 1280 ± | 233 ± | NA | 0.0266 | 0.000708 |
| | 0.188 | 72.2 | 14.7 | | | |
| Non- | 4.35 ± | 1110 ± | 173 ± | −380 ± | 0.0246 | 0.000607 |
| competitive | 0.205 | 77.5 | 19.2 | 130 | | |
| Un-competitive | 7.33 ± | 2320 ± | NA | 83.9 ± | 0.0506 | 0.00256 |
| | 0.924 | 352 | | 17.0 | | |

^{a}The data presented in FIG. 9, right panel, were analyzed using the program KinetAsyst, which uses an adaptation of the algorithm of Cleland. The data were fit to the indicated inhibition mechanisms and the values of the key parameters are shown. Concentrations are in μM.
NA indicates not applicable.

TABLE 7
Inhibition of PtxD by sulfite with NAD constant at 50 μM^{a}.

| Mechanism | Vₘₐₓ | K_{M} | Kᵢₛ | Kᵢᵢ | Sigma | Variance |
|---|---|---|---|---|---|---|
| Competitive | 10.6 ± | 2060 ± | 16.1 ± | NA | 0.0211 | 0.000443 |
| | 2.53 | 547 | 0.645 | | | |
| Non- | 16.5 ± | 3910 ± | 21.1 ± | 4.97 ± | 0.0197 | 0.000389 |
| competitive | 9.50 | 2060 | 2.11 | 3.53 | | |
| Un-competitive | 36900 ± | −200000 ± | NA | −9.00 ± | 25.1 | 631 |
| | 3420000 | 9220000 | | 70.1 | | |

^{a}The data presented in FIG. 10, left panel, were analyzed using the program KinetAsyst, which uses an adaptation of the algorithm of Cleland. The data were fit to the indicated inhibition mechanisms and the values of the key parameters are shown. Concentrations are in μM.
NA indicates not applicable.

TABLE 8
Inhibition of PtxD by sulfite with phosphite constant at 50 μM^{a}.

| Mechanism | Vₘₐₓ | K_{M} | Kᵢₛ | Kᵢᵢ | Sigma | Variance |
|---|---|---|---|---|---|---|
| Competitive | 0.445 ± | 32.3 ± | −8.212 × 10²¹ | NA | 0.188 | 0.0354 |
| | 0.0671 | 17.2 | ±1.36 × 10⁴² | | | |
| Non- | 3.78 ± | 695 ± | 241 ± | 1.15 ± | 0.01180 | 0.000140 |
| competitive | 0.238 | 55.0 | 181 | 0.0807 | | |
| Un- | 4.04 ± | 729 ± | NA | 10.8 ± | 0.0119 | 0.000141 |
| competitive | 0.235 | 53.9 | | 0.0630 | | |

^{a}The data presented in FIG. 10, right panel, were analyzed using the program KinetAsyst, which uses an adaptation of the algorithm of Cleland. The data were fit to the indicated inhibition mechanisms and the values of the key parameters are shown. Concentrations are in μM.
NA indicates not applicable.

TABLE 9
Reaction conditions and results for cofactor regeneration assays.

| Enzyme | PtxD | [Pt] | [S]^{a} | [NAD⁺] | Vol | TTN | TN | TTN | TN |
|---|---|---|---|---|---|---|---|---|---|
| (Units) | (U and moles) | (mM) | (mM) | (mM) | (mL) | (NAD⁺) | (NAD⁺) | (PtxD) | (PtxD) |
| LLDH | 0.58, | 500 | 200 | 0.1 | 2.0 | 2000 | 12 | 9.8 × 10⁴ | 612 |
| (0.39) | (4 × 10⁻⁹) | | | | | | | | |
| HLADH | 0.44, | 500 | 200 | 0.1 | 1.5 | 2000 | 123 | 1.6 × 10⁵ | 9846 |
| (0.29) | (2.5 × 10⁻⁹) | | | | | | | | |

^{a}Substrate = pyruvate and acetaldehyde for LLDH and HLADH, respectively.

We claim:

1. A purified enzyme capable of converting phosphite to phosphate, wherein the enzyme is a phosphite dehydrogenase produced by a recombinant process.

2. The enzyme of claim 1, wherein the enzyme is about 95% pure or greater.

3. The enzyme of claim 1, wherein the enzyme is purified to homogeneity.

4. A purified enzyme capable of converting phosphite to phosphate, wherein the enzyme is a phosphite dehydrogenase isolated from a natural source.

5. The enzyme of claim 4, wherein the enzyme is about 90% pure or greater.

6. A method of purifying a phosphite dehydrogenase enzyme comprising the steps of:
(a) contacting a solution of the enzyme with a first NAD affinity column incapable of binding the enzyme, and eluting the enzyme as a solution having fewer impurities; and
(b) contacting the resulting eluent with a second NAD affinity column capable of binding the enzyme, and eluting the enzyme as a solution.

7. The method of claim 6, wherein the second NAD affinity column is characterized by attachment of the ligand at N-6.

8. The method of claim 6, wherein the first NAD affinity column is characterized by attachment of the ligand at C-8.

9. A method of preparing NADH or NADPH comprising the step of:
contacting a solution of NAD or NADP with a phosphite dehydrogenase enzyme and phosphite.

10. A method of reducing NADH or NADPH having an isotope of hydrogen comprising the step of:
contacting a solution of NAD or NADP with a phosphite dehydrogenase enzyme and phosphite; where the phosphite includes an isotope of hydrogen.

11. A method of oxidizing phosphite to phosphate comprising the step of:
contacting a solution of phosphite with a phosphite dehydrogenase enzyme and an oxidizing agent selected from the group consisting of NAD and NADP.

12. A method of selectively oxidizing phosphite to phosphate comprising the step of:
contacting a solution of phosphite with a phosphite dehydrogenase enzyme and an oxidizing agent selected from the group consisting of NAD and NADP, where said solution contains at least one other oxidizable species selected from the group consisting of hypophosphite, methylphosphonate, arsenite, and nitrite.

13. A method of reducing a compound to an overall lower oxidation state comprising the steps of:
(a) contacting the compound with a first oxidoreductase enzyme that uses a cofactor selected from the group consisting of NADH and NADPH; and
(b) contacting the compound with a phosphite dehydrogenase enzyme, phosphite, and an agent selected from the group consisting of NAD and NADP.

14. The method of claim 13, wherein the oxidoreductase enzyme is selected from the group consisting of formate dehydrogenase, glucose dehydrogenase, L-lactate dehydrogenase, D-lactate dehydrogenase, malate dehydrogenase, horse liver alcohol dehydrogenase, and aldehyde dehydrogenase.

15. The method of claim 13, wherein the cofactor is NADH, and the agent is NAD.

16. A method of reducing a compound to an overall lower oxidation state, where the reduction includes introducing an isotope of hydrogen, comprising the steps of:
(a) contacting the compound with a first oxidoreductase enzyme that uses a cofactor selected from the group consisting of NADH and NADPH; and
(b) contacting the compound with a phosphite dehydrogenase enzyme, phosphite, and an agent selected from the group consisting of NAD and NADP; where the phosphite includes the isotope of hydrogen.

17. The method of claim 16, wherein the oxidoreductase enzyme is selected from the group consisting of formate dehydrogenase, glucose dehydrogenase, L-lactate dehydrogenase, D-lactate dehydrogenase, malate dehydrogenase, horse liver alcohol dehydrogenase, and aldehyde dehydrogenase.

18. The method of claim 16, wherein the cofactor is NADH, and the agent is NAD.

19. A method of stereoselectively reducing a prochiral compound to an overall lower oxidation state comprising the step of:
contacting the prochiral compound with a mixture comprising (1) an oxidoreductase enzyme that uses a cofactor selected from the group consisting of NADH and NADPH, and (2) a phosphite dehydrogenase enzyme, phosphite, and an agent selected from the group consisting of NAD and NADP; where the compound is reduced at the prochiral center to form a chiral compound, and a solution of the chiral compound is optically active.

20. The method of claim 19, wherein the oxidoreductase enzyme is selected from the group consisting of formate dehydrogenase, glucose dehydrogenase, L-lactate dehydrogenase, D-lactate dehydrogenase, malate dehydrogenase, horse liver alcohol dehydrogenase, and aldehyde dehydrogenase.

21. The method of claim 19, wherein the cofactor is NADH, and the agent is NAD.

22. A method of stereoselectively reducing a prochiral compound to an overall lower oxidation state, where the reduction includes introducing an isotope of hydrogen, comprising the step of:
contacting the prochiral compound with a mixture comprising (a) an oxidoreductase enzyme that uses a cofactor selected from the group consisting of NADH and NADPH, and (b) a phosphite dehydrogenase enzyme, phosphite,and an agent selected from the group consisting of NAD and NADP; where the phosphite includes the isotope of hydrogen; and the compound is reduced at the prochiral center to form a chiral compound, and a solution of the chiral compound is optically active.

23. The method of claim 22, wherein the oxidoreductase enzyme is selected from the group consisting of form ate dehydrogenase, glucose dehydrogenase, L-lactate dehydrogenase, D-lactate dehydrogenase, malate dehydrogenase, horse liver alcohol dehydrogenase, and aldehyde dehydrogenase.

24. The method of claim 22, wherein the cofactor is NADH, and the agent is NAD.

25. A purified enzyme capable of converting phosphite to phosphate, where the enzyme comprises the sequence GWX₁PX₂X₃YX₄X₅GL, where X₁ is R, Q, T, or K; X₂ is A, V, Q, R, K, or H; X₃ is L or F; X₄ is G or F; and X₅ is T, R, M, or L.

26. The enzyme of claim 25, wherein X₃ is F.

27. The enzyme of claim 25, wherein the enzyme comprises a sequence selected form the group consisting of GWRPQFYSLGL, GWRPHLYGMGL, GWKPKLYSRGL, GWTPRFYGTGL, GWQPQFYGTGL, GWQPQFYGTGL, GWQPQFYGTGL, GWQPQFYGTGL, GWQPQFYGTGL, GWQPQFYGTGL, GWRPVLYGTGL, and GWRPALYGTGL

28. The enzyme of claim 25, wherein the enzyme includes a NAD binding sequence.

29. The enzyme of claim 28, wherein the NAD binding sequence is GX₁GX₂X₃GX₄AX₅X₆X₇RL, where X₁ is M, T, or L; X₂ is K, S, or A; X₃ is V, I, or L; X₄ is Q, L, R, or K; X₅ is I, M, V, or L; X₆ is L or A; and X₇ is A, H, E, D, K, Q, or G.

30. The enzyme of claim 29, wherein X₃ is I, and X₅ is M.

31. The enzyme of claim 28, wherein the NAD binding sequence is selected from the group consisting of GMGALGKAIAGRL, GMGSLGQALAQRL, GMGKLGKALAQRL, GTGAVGRAVAKRL, GMGAIGLAMADRL, GMGAIGLAMADRL, GMGAIGLAMADRL, GMGAIGLAMAERL, GMGAIGQAMAERL, GMGAIGQAMAERL, GMGAVGQAIAHRL, GLGKVGQAILARL.

32. The enzyme of claim 25, wherein the enzyme is produced by a recombinant process.

33. The enzyme of claim 32, wherein the enzyme is about 95% pure or greater.

34. The enzyme of claim 32, wherein the enzyme is purified to homogeneity.

35. The enzyme of claim 25, wherein the enzyme is produced by a naturally-occurring organism.

36. The enzyme of claim 35, wherein the enzyme is about 90% pure or greater.

37. A protein comprising the amino acid sequence:
MKPRIVTTHRIHPDTLALLETAAEVISNQSDSTMSREEVLLRTNDADGM
VFMPDSIDADFLSACPNLKVIGAALKGYDNFDVEACTRHGIWFTIVPDLL
TSPTAELTIGLLLSITRNMLQGDNYIRSRQFNGWTPRFYGTGLTGKTAGI
IGTGAVGRAVAKRLAAFDMQIQYTDPQPLPQESERAWNASRTSLDWLLAT
SDFIIPMLPMSSDTHHTINARALDRMKPGAYLVNACRGSIVDERAVVHAL
RTGHLGGYAADVFEMEEWARPDRPHSIPDELLDPALPTFFTPHLGSAVKS
VRMEIEREAALSILEALQGRIPRGAVNHVGAGR.

38. An isolated nucleic acid that encodes the protein of claim 37.

39. The nucleic acid of claim 37, wherein the nucleic acid comprises the nucleotide sequence:
ATGAAACCAAGAATCGTAACAACGCATCGAATCCACCCTGACACGCTGGC
CCTCCTTGAGACCGCCGCTGAAGTAATTTCCAATCAGTCCGATTCAACCA
TGTCGCGGGAAGAGGTACTGTTGCGCACCAATGATGCGGACGGGATGATG
GTGTTTATGCCGGATAGCATAGATGCGGATTTTCTATCCGCGTGCCCCAA
TCTGAAGGTCATCGGCGCCGCGCTTAAAGGATATGACAACTTTGATGTCG
AGGCATGTACCCGCCATGGGATTTGGTTTACGATTGTTCCTGATTTGCTT
ACGAGTCCCACAGCTGAACTAACGATTGGCCTGTTGCTGAGCATCACACG
GAATATGCTGCAAGGTGATAATTACATTCGATCACGCCAGTTCAATGGTT
GGACCCCGCGGTTTTATGGCACAGGTTTGACGGGTAAAACCGCCGGCATC
ATTGGGACGGGAGCGGTCGGGCGGGCGGTCGCAAAGCGGCTGGCCGCCTT
CGATATGCAAATTCAGTACACGGATCCGCAGCCTTTGCCGCAAGAGTCGG
AAAGGGCATGGAATGCGAGCAGAACATCGCTGGACCAGCTATTGGCGACA
AGCGATTTCATCATTCCCATGCTGCCGATGTCGTCAGATACCCACCACAC
CATAAATGCTCGGGCATTAGACCGCATGAAGCCCGGTGCGTACCTCGTCA
ATGCCTGTCGCGGCTCCATCGTAGATGAGCGGGCCGTGGTGCATGCGCTT
CGGACGGGGCATCTGGGCGGTTACGCCGCAGACGTCTTCGAGATGGAGGA
ATGGGCGCGTCCCGACAGGCCGCATTCTATTCCTGACGAATTGCTTGATC
CTGCTTTACCCACATTCTTTACGCCTCACCTGGGTTCGGCGGTCAAATCG
GTACGGATGGAAATCGAGCGTGAAGCCGCCCTCAGTATCCTCGAAGCGTT
GCAAGGGCGCATTCCACGCGGAGCGGTCAACCATGTTGGAGCGGGGAGAT
GACTGA.

40. A protein comprising the amino acid sequence:
MARKTIVTNWVHPEVLDLLSTRGPAEANTTREPWPRDEIIRRAHGADAML
AFMTDHVDAAFLDACPELKIVACALKGADNFDMEACRARKVAVTIVPDLL
TAPTAELAVGLMITLGRNLLAGDRLIRERPFAGWRPVLYGTGLDGAEVGI
VGMGAVGQAIAHRLRPFRCRLSYCDARPLSPAAEDAQGLLRRDLADLVAR
SDYLVLALPLTPASRHLIDAAALAGMKPGALLINPARGSLVDEAAVADAL
EAGHLGGYAADVFETEDWARPDRPAAIEARLLAHPRTVLTPHIGSAVDSV
RRDIALAAARDILRHLDGLQQDPPSRDRSAG.

41. An isolated nucleic acid that encodes the protein of claim 40.

42. The nucleic acid of claim 41, wherein the nucleic acid comprises the nucleotide sequence:
ATGGCCCGCAAGACCATCGTCACCAATTGGGTGCACCCCGAGGTGCTGGA
CCTGCTTTCCACCCGCGGCCCGGCGGAGGCCAACACCACCCGCGAACCGT
GGCCGCGCGACGAGATCATCCGCCGCGCCCACGGCGCGGACGCCATGCTC
GCCTTCATGACCGACCATGTGGACGCCGCCTTCCTCGACGCCTGCCCGGA
GCTGAAGATCGTCGCCTGCGCCCTGAAGGGGGCCGACAATTTCGACATGG
AGGCGTGCCGTGCCCGCAAGGTGGCGGTGACCATCGTGCCGGACCTGCTC
ACCGCGCCGACGGCGGAGCTGGCGGTGGGCCTCATGATCACCCTCGGCCG
CAACCTGCTCGCCGGCGACCGGCTGATCCGCGAACGGCCGTTCGCCGGCT
GGCGGCCGGTGTTGTACGGCACCGGCCTCGACGGGGCGGAGGTGGGCATC
GTCGGCATGGGCGCGGTGGGGCAGGCCATCGCCCACCGCCTGCGGCCGTT
CCGCTGCCGCCTCTCCTATTGCGACGCGCGGCCCCTCTCCCCCGCAGCAG
AAGATGCCCAGGGGCTGCTGCGGCGCGACCTCGCCGACCTCGTCGCGCGC
AGCGACTACCTGGTGCTGGCCCTGCCGCTGACACCCGCCTCGCGCCACCT
GATCGACGCCGCGGCCCTTGCGGGCATGAAGCCGGGCGCGCTGCTCATCA
ATCCCGCCAGGGGCTCGCTGGTGGACGAGGCGGCGGTGGCCGATGCCCTG
GAGGCCGGCCATCTCGGCGGCTATGCGGCCGATGTGTTCGAAACCGAGGA
CTGGGCCCGCCCCGATCGCCCCGCCGCCATCGAGGCGCGCCTCCTCGCCC
ATCCGCGCACGGTGCTCACCCCGCACATCGGATCGGCGGTGGACAGCGTG
CGGCGGGACATCGCGCTTGCCGCGGCGCGGGACATCCTGCGCCATCTGGA
CGGATTGCAGCAGGACCCGCCTTCGCGCGATCGTTCGGCGGCCTGA.

43. A protein comprising the amino acid sequence:
MLPKLVITHRVHDEILQLLAPHCELMTNQSDSTLTREEILRRCRDAQAMM
AFMPDRVDAEFLQACPELRVVGCALKGFDNFDVDACTARGVWLTFVPDLL
TVPTAELAIGLAVGLGRHLRAADAFVRSGEFQGWQPQFYGTGLDNATVGI
LGMGAIGQAMAERLQGWGATLQYHEAKALDTQTEQRLGLRQVACSELFAS
SDFILLALPLNADTEHLVNAELLALVRPGALLVNPCRGSVVDEAAVLAAL
ERGQLGGYAADVFEMEDWARADRPRLIDPALLAHPNTLFTPHIGSAVRAV
RLEIERCAAQSIIQALAGARPINAANRLPQAEPAAC.

44. An isolated nucleic acid that encodes the protein of claim 43.

45. A protein comprising the amino acid sequence:
MLPKLVITHRVHDEILQLLAPHCELMTNQTDSTLTREEILRRCRDAQAMM
AFMPDRVDADFLQACPELRVVGCALKGFDNFDVDACTARGVWLTFVPDLL
TVPTAELAIGLAVGLGRHLRAADAFVRSGEFQGWQPQFYGTGLDNATVGI
LGMGAIGLAMADRLQGWGATLQYHEAKALDTQTEQRLGLRQVACSELFAS
SDFILLALPLNADTQHLVNAELLALVRPGALLVNPCRGSVVDEAAVLAAL
ERGQLGGYAADVFEMEDWARADRPRLIDPALLAHPNTLFTPHIGSAVRAV
RLEIERCAAQNIIQVLAGARPINAANRLPKAEPAAC.

46. An isolated nucleic acid that encodes the protein of claim 45.

47. The nucleic acid of claim 46, wherein the nucleic acid comprises the nucleotide sequence:
ATGCTGCCGAAACTCGTTATAACTCACCGAGTACACGATGAGATCCTGCA
ACTGCTGGCGCCACATTGCGAGCTGATGACCAACCAGACCGACAGCACGC
TGACGCGCGAGGAAATTCTGCGCCGCTGTCGCGATGCTCAGGCGATGATG
GCGTTCATGCCCGATCGGGTCGATGCAGACTTTCTTCAAGCCTGCCCTGA
GCTGCGTGTAGTCGGCTGCGCGCTCAAGGGCTTCGACAATTTCGATGTGG
ACGCCTGTACTGCCCGCGGGGTCTGGCTGACCTTCGTGCCTGATCTGTTG
ACGGTCCCGACTGCCGAGCTGGCGATCGGACTGGCGGTGGGGCTGGGGCG
GCATCTGCGGGCAGCAGATGCGTTCGTCCGCTCTGGCGAGTTCCAGGGCT
GGCAACCACAGTTCTACGGCACGGGGCTGGATAACGCTACGGTCGGCATC
CTTGGCATGGGCGCCATCGGACTGGCCATGGCTGATCGCTTGCAGGGATG
GGGCGCGACCCTGCAGTACCACGAGGCGAAGGCTCTGGATACACAAACCG
AGCAACGGCTCGGCCTGCGCCAGGTGGCGTGCAGCGAACTCTTCGCCAGC
TCGGACTTCATCCTGCTGGCGCTTCCCTTGAATGCCGATACCCAGCATCT
GGTCAACGCCGAGCTGCTTGCCCTCGTACGGCCGGGCGCTCTGCTTGTAA
ACCCCTGTCGTGGTTCGGTAGTGGATGAAGCCGCCGTGCTCGCGGCGCTT
GAGCGAGGCCAGCTCGGCGGGTATGCGGCGGATGTATTCGAAATGGAAGA
CTGGGCTCGCGCGGACCGGCCGCGGCTGATCGATCCTGCGCTGCTCGCGC
ATCCGAATACGCTGTTCACTCCGCACATAGGGTCGGCAGTGCGCGCGGTG
CGCCTGGAGATTGAACGTTGTGCAGCGCAGAACATCATCCAGGTATTGGC
AGGTGCGCGCCCAATCAACGCTGCGAACCGTCTGCCCAAGGCCGAGCCTG
CCGCATGTTGA.

48. A protein comprising the amino acid sequence:
MLPKLVITHRVHDEILQLLAPHCELMTNQTDSTLTREEILRRCRDAQAMM
AFMPDRVDADFLQACPELRVVGCALKGFDNFDVDACTARGVWLTFVPDLL
TVPTAELAIGLAVGLGRHLRAADAFVRSGEFQGWQPQFYGTGLDNATVGI
LGMGAIGLAMADRLQGWGATLQYHEAKALDTQTEQRLGLRQVACSELFAS
SDFILLALPLNADTQHLVNAELLALVRPGALLVNPCRGSVVDEAAVLAAL
ERGQLGGYAADVFEMEDWARADRPRLIDPALLAHPNTLFTPHIGSAVRAV
RLEIERCAAQNIIQVLAGARPINAANRLPKAEPAAC.

49. An isolated nucleic acid that encodes the protein of claim 48.

50. The nucleic acid of claim 49, wherein the nucleic acid comprises the nucleotide sequence:
ATGCTGCCGAAACTCGTTATAACTCACCGAGTACACGATGAGATCCTGCA
ACTGCTGGCGCCACATTGCGAGCTGATGACCAACCAGACCGACAGCACGC
TGACGCGCGAGGAAATTCTGCGCCGCTGTCGCGATGCTCAGGCGATGATG
GCGTTCATGCCCGATCGGGTCGATGCAGACTTTCTTCAAGCCTGCCCTGA
GCTGCGTGTAGTCGGCTGCGCGCTCAAGGGCTTCGACAATTTCGATGTGG
ACGCCTGTACTGCCCGCGGGGTCTGGCTGACCTTCGTGCCTGATCTGTTG
ACGGTCCCGACTGCCGAGCTGGCGATCGGACTGGCGGTGGGGCTGGGGCG
GCATCTGCGGGCAGCAGATGCGTTCGTCCGCTCTGGCGAGTTCCAGGGCT
GGCAACCACAGTTCTACGGCACGGGGCTGGATAACGCTACGGTCGGCATC
CTTGGCATGGGCGCCATCGGACTGGCCATGGCTGATCGCTTGCAGGGATG
GGGCGCGACCCTGCAGTACCACGAGGCGAAGGCTCTGGATACACAAACCG
AGCAACGGCTCGGCCTGCGCCAGGTGGCGTGCAGCGAACTCTTCGCCAGC
TCGGACTTCATCCTGCTGGCGCTTCCCTTGAATGCCGATACCCAGCATCT
GGTCAACGCCGAGCTGCTTGCCCTCGTACGGCCGGGCGCTCTGCTTGTAA
ACCCCTGTCGTGGTTCGGTAGTGGATGAAGCCGCCGTGCTCGCGGCGCTT
GAGCGAGGCCAGCTCGGCGGGTATGCGGCGGATGTATTCGAAATGGAAGA
CTGGGCTCGCGCGGACCGGCCGCGGCTGATCGATCCTGCGCTGCTCGCGC
ATCCGAATACGCTGTTCACTCCGCACATAGGGTCGGCAGTGCGCGCGGTG
CGCCTGGAGATTGAACGTTGTGCAGCGCAGAACATCATCCAGGTATTGGC
AGGTGCGCGCCCAATCAACGCTGCGAACCGTCTGCCCAAGGCCGAGCCTG
CCGCATGTTGA.

51. A protein comprising the amino acid sequence:
MLPKLVITHRVHDEILQLLAPHCELMTNQSDSTLTREEILRRCRDAQAMM
AFMPDRVDAEFLQACPELRVVGCALKGFDNFDVDACTARGVWLTFVPDLL
TVPTAELAIGLAVGLGRHLRAADAFVRSGEFQGWQPQFYGTGLDNATVGI
LGMGAIGQAMAERLQGWGATLQYHEAKALDTQTEQRLGLRQVACSELFAS
SDFILLALPLNADTEHLVNAELLALVRPGALLVNPCRGSVVDEAAVLAAL
ERGQLGGYAADVFEMEDWARADRPRLIDPALLAHPNTLFTPHIGSAVRAV
RLEIERCAAQSIIQALAGARPINAANRLPQAEPAAC.

52. An isolated nucleic acid that encodes the protein of claim 51.

53. The nucleic acid of claim 52, wherein the nucleic acid comprises the nucleotide sequence:
ATGCTGCCGAAACTCGTTATAACTCACCGAGTACACGATGAGATCCTGCA
ACTGCTGGCGCCACATTGCGAGCTGATGACCAACCAGAGCGACAGCACGC
TGACGCGCGAGGAAATTCTGCGCCGCTGCCGCGATGCTCAGGCGATGATG
GCGTTCATGCCCGATCGGGTCGATGCAGAATTTCTCCAAGCCTGCCCCGA
GCTGCGTGTGGTCGGCTGCGCGCTCAAGGGCTTCGACAATTTCGATGTGG
ACGCCTGTACTGCCCGCGGGGTCTGGTTGACCTTCGTGCCTGATCTGTTG
ACGGTCCCGACTGCCGAGCTGGCGATCGGACTGGCGGTGGGGCTGGGGCG
GCATCTGCGCGCAGCAGATGCGTTCGTCCGCTCTGGCGAGTTCCAGGGCT
GGCAACCACAGTTCTACGGCACGGGGCTGGATAACGCGACGGTCGGCATC
CTTGGCATGGGCGCCATCGGACAGGCCATGGCTGAGCGCTTGCAGGGATG
GGGCGCGACCCTGCAGTACCACGAGGCGAAGGCTCTGGATACACAAACCG
AGCAACGGCTCGGCCTGCGCCAGGTGGCGTGCAGCGAACTCTTCGCCAGC
TCGGACTTCATCCTGCTGGCGCTTCCCTTGAATGCCGATACTGAGCATCT
GGTCAACGCCGAGCTGCTTGCCCTCGTACGGCCGGGCGCTCTGCTTGTAA
ACCCCTGTCGTGGTTCGGTAGTGGATGAAGCCGCCGTGCTCGCGGCGCTT
GAGCGAGGCCAGCTCGGCGGGTATGCGGCGGATGTATTCGAAATGGAAGA
CTGGGCTCGCGCGGACCGGCCGCGGCTGATCGATCCTGCGCTGCTCGCGC
ATCCGAATACGCTCTTCACTCCGCACATAGGGTCGGCAGTGCGCGCGGTG
CGCCTGGAGATTGAACGTTGTGCAGCGCAGAGCATCATCCAGGCGTTGGC
AGGTGCGCGCCCAATCAACGCTGCGAACCGTCTGCCCCAGGCCGAGCCTG
CCGCATGTTGA.

54. A protein comprising the amino acid sequence:
MKPKVVITHWVHPEIITNLSEYCEVVANPTRETLPREEILKLAQDAEALM
VFMPDRIDEAFLKACPKLKIIAGALKGYDNFDVDACTRQGIWFTIVPSLL
AVPTAELTIGLIIGLARQMLLGDRLIRQGTFAGWRPHLYGMGLANRTLGI
VGMGSLGQALAQRLSSFEMNLIYTDAIPLPKEKAAAWCLSQVSLDTLLAT
SDFVVLMVPLQPETFHLINEKSLARMKPGSFLINPCRGSVVDEQAVSDAL
ASGHLAGYAADVFELEDWARSDRPSKIPPSLLEKQDQTFFTPHLGSAVDD
LRYDIAIEASQNILQVLQGHSPQGAINCPS.

55. An isolated nucleic acid that encodes the protein of claim 54.

56. A protein comprising the amino acid sequence:
MKPKVVITNWVHPEVIELLKPSCEVIANPSKEALSREEILQRAKDAEALM
VFMPDTIDEAFLRECPKLKIIAAALKGYDNFDVAACTHRGIWFTIVPSLL
SAPTAEITIGLLIGLGRQMLEGDRFIRTGKFTGWRPQFYSLGLANRTLGI
VGMGALGKAIAGRLAGFEMQLLYSDPVALPPEQEATGNISRVPFETLIES
SDFVVLVVPLQPATLHLINANTLAKMKPGSFLINPCRGSVVDEQAVCKAL
ESGHLAGYAADVFEMEDWYRSDRPHNIPQPLLENTKQTFFTPHIGSAVDE
LRHNIALEAAQNILQALQGQKPQGAVNYLRES.

57. An isolated nucleic acid that encodes the protein of claim 56.

58. A protein comprising the amino acid sequence:
MDKKPLVVITHWIHSQVIDYLSKTCEVILNQTQYTLSREEIINRTKEAEG
LMVFMPDFIESSFLDACPQLKVIAGALRGYDNFDIDACTKRGIWFTIVPD
LLAAPTAELTVGLLLGLARRMLEGDRLIRDGQFQGWKPKLYSRGLLHQNL
GIVGMGKLGKALAQRLVGFNMNLFYSDSIPLSVEQETAWKISRVSLEELL
KISDYVVLMVPLIPDTYHLINQDTLAKMKPKSFLINACRGSVVDEKAIGS
AIQSGHLAGYAAVDFEMEDRAIPHRPQGIYNTLITEKNKTFLTPHLGSAV
DEIRHDISFEAAKNIVQVLSGEIPQGAVNKL.

59. An isolated nucleic acid that encodes the protein of claim 58.

60. A protein comprising the amino acid sequence:
MNHRKIVVTQPVHEEVLRKLQAEGEVIMNPGPDPWSPSQLREYLVDADAM
MAFMTDSVTKESLLNAPRLKTISCALKGYDNFDLRACAQAGVSVTFVPDL
LTEPTAELAIGLAIAAGRNVLQGDAATRAGYSGWRPALYGTGLHGSVASV
IGLGKVGQAILARLAGFGCARLLGVDPSVRLDQVELVTLDEAVSTSDYVF
LAVPLVSDTRHLVDSRMLQLSKKGQILVNVGRGSVVDERAVVDALANEQL
GAYAADVYEMEDWLLPDRPREIHPGLTNNARTVLTPHIGSAVRRVRFEIE
MRAAENLVRSLRGESLSDVAVEASAAA.

61. An isolated nucleic acid that encodes the protein of claim 60.

62. A protein comprising the amino acid sequence:
MLPKLVITHRVHDEILQLLAPHCELVTNQTDSTLTREEILRRCRDAQAMM
AFMPDRVDADFLQACPELRVVGCALKGFDNFDVDACTARGVWLTFVPDLL
TVPTAELAIGLAVGLGRHLRAADAFVRSGEFQGWQPQFYGTGLDNATVGI
LGMGAIGLAMAERLQGWGATLQYHEAKALDTQTEQRLGLRQVACSELFAS
SDFILLALPLNADTQHLVNAELLALVRPGALLVNPCRGSVVDEAAVLAAL
ERGQLGGYAADVFEMEDWARADRPRLIDPALLAHPNTLFTPHIGSAVRAV
RLEIERCAAQNIIQVLAGARPINAANRLPKAEPAAC.

63. An isolated nucleic acid that encodes the protein of claim 62.

64. A purified enzyme capable of converting phosphite to phosphate, where the enzyme comprises a phosphite catalytic site including a histidine, a glutamate, and an arginine.

65. The enzyme of claim 64, wherein the enzyme includes a NAD binding sequence.

66. The enzyme of claim 65, wherein the NAD binding sequence is GX₁GX₂X₃GX₄AX₅X₆X₇RL, where X₁ is M, T, or L; X₂ is K, S, or A; X₃ is V, I, or L; X₄ is Q, L, R, or K; X₅ is I, M, V, or L; X₆ is L or A; and X₇ is A, H, E, D, K, Q, or G.

67. The enzyme of claim 66, wherein X₃ is I, and X₅ is M.

68. The enzyme of claim 64, wherein the NAD binding sequence is selected from the group consisting of GMGALGKAIAGRL, GMGSLGQALAQRL, GMGKLGKALAQRL, GTGAVGRAVAKRL, GMGAIGLAMADRL, GMGAIGLAMADRL, GMGAIGLAMADRL, GMGAIGLAMAERL, GMGAIGQAMAERL, GMGAIGQAMAERL, GMGAVGQAIAHRL, GLGKVGQAILARL.

69. The enzyme of claim 64, wherein the enzyme is produced by a recombinant process.

70. The enzyme of claim 69, wherein the enzyme is about 95% pure or greater.

71. The enzyme of claim 69, wherein the enzyme is purified to homogeneity.

72. The enzyme of claim 64, wherein the enzyme is produced by a naturally-occurring organism.

73. The enzyme of claim 72, wherein the enzyme is about 90% pure or greater.


SEQUENCE LISTING (62 sequences)

SEQ ID NO: 1 - protein, 336 aa, Pseudomonas stutzeri
  strain WM88
MLPKLVITHRVHDEILQLLAPHCELMTNQTDSTLTREEILRRCRDAQAMMAFMPDRVDADFLQACPELRVVGCALKGFDNFDVDACTARGVWLTFVPDLLTVPTAELAIGLAVGLGRHLRAADAFVRSGEFQGWQPQFYGTGLDNATVGILGMGAIGLAMADRLQGWGATLQYHEAKALDTQTEQRLGLRQVACSELFASSDFILLALPLNADTQHLVNAELLALVRPGALLVNPCRGSVVDEAAVLAALERGQLGGYAADVFEMEDWARADRPRLIDPALLAHPNTLFTPHIGSAVRAVRLEIERCAAQNIIQVLAGARPINAANRLPKAEPAAC

SEQ ID NO: 2 - protein, 400 aa, Pseudomonas sp.
  strain 101
AKVLCVLYDDPVDGYPKTYARDDLPKIDHYPGGQTLPTPKAIDFTPGQLLGSVSGELGLRKYLESNGHTLVVTSDKDGPDSVFERELVDADVVISQPFWPAYLTPERIAKAKNLKLALTAGIGSDHVDLQSAIDRNVTVAEVTYCNSISVAEHVVMMILSLVRNYLPSHEWARKGGWNIADCVSHAYDLEAMHVGTVAAGRIGLAVLRRLAPFDVHLHYTDRHRLPESVEKELNLTWHATREDMYPVCDVVTLNCPLHPETEHMINDETLKLFKRGAYIVNTARGKLCDRDAVARALESGRLAGYAGDVWFPQPAPKDHPWRTMPYNGMTPHISGTTLTAQARYAAGTREILECFFEGRPIRDEYLIVQGGALAGTGAHSYSKGNATGGSEEAAKFKKAV

SEQ ID NO: 3 - protein, 336 aa, Lactobacillus helveticus
TKVFAYAIRKDEEPFLNEWKEAHKDIDVDYTDKLLTPETAKLAKGADGVVVYQQLDYTADTLQALADAGVTKMSLRNVGVDNIDMDKAKELGFQITNVPVYSPNAIAEHAAIQAARVLRQDKRMDEKMAKRDLRWAPTIGREVRDQVVGVVGTGHIGQVFMRIMEGFGAKVIAYDIFKNPELEKKGYYVDSLDDLYKQADVISLHVPDVPANVHMINDKSIAEMKDGVVIVNCSRGRLVDTDAVIRGLDSGKIFGFVMDTYEDEVGVFNKDWEGKEFPDKRLADLIDRPNVLVTPHTAFYTTHAVRNMVVKAFNNNLKLINGEKPDSPVALNKNKF

SEQ ID NO: 4 - protein, 321 aa, Hyphomicrobium methylovorum
  strain GM2
SKKKILITWPLPEAAMARARESYDVIAHGDDPKITIDEMIETAKSVDALLITLNEKCRKEVIDRIPENIKCISTYSIGFDHIDLDACKARGIKVGNAPHGVTVATAEIAMLLLLGSARRAGEGEKMIRTRSWPGWEPLELVGEKLDNKTLGIYGFGSIGQALAKRAQGFDMDIDYFDTHRASSSDEASYQATFHDSLDSLLSVSQFFSLNAPSTPETRYFFNKATIKSLPQGAIVVNTARGDLVDNELVVAALEAGRLAYAGFDVFAGEPNINEGYYDLPNTFLFPHIGSAATQAREDMAHQANDLIDALFGGADMSYALA

SEQ ID NO: 5 - protein, 360 aa, Escherichia coli
AKVSLEKDKIKFLLVEGVHQKALESLRAAGYTNIEFHKGALDDEQLKESIRDAHFIGLRSRTHLTEDVINAAEKLVAIGCFCIGTNQVDLDAAAKRGIPVFNAPFSNTRSVAELVIGELLLLLRGVPEANAKAHRGVWNKLAAGSFEARGKKLGIIGYGHIGTQLGILAESLGMYVYFYDIENKLPLGNATQVQHLSDLLNMSDVVSLHVPENPSTKNMMGAKEISLMKPGSLLINASRGTVVDIPALCDALASKHLAGAAIDVFPTEPATNSDPFTSPLCEFDNVLLTPHIGGSTQEAQENIGLEVAGKLIKYSDNGSTLSAVNFPEVSLPLHGGRRLMHIHENRPGVLTALNKIFAEQ

SEQ ID NO: 6 - protein, 323 aa, Escherichia coli
MKILVDENMPYARDLFSRLGEVTAVPGRPIPVAQLADADALMVRSVTKVNESLLAGKPIKFVGTATAGTDHVDEAWLKQAGIGFSAAPGCNAIAVVEYVFSSLLMLAERDGFSLYDRTVGIVGVGNVGRRLQARLEALGIKTLLCDPPRADRGDEGDFRSLDELVQRADILTFHTPLFKDGPYKTLHLADEKLIRSLKPGAILINACRGAVVDNTALLTCLNEGQKLSVVLDVWEGEPELNVELLKKVDIGTSHIAGYTLEGKARGTTQVFEAYSKFIGHEQHVALDTLLPAPEFGRITLHGPLDQPTLKRLVHLVYDVRRDD

SEQ ID NO: 7 - protein, 333 aa, Lactobacillus casei
MKIIAYGARVDEIQYFKQWAKDTGNTLEYHTEFLDENTVEWAKGFDGINSLQTTPYAAGVFEKMHAYGIKFLTIRNVGTDNIDMTAMKQYGIRLSNVPAYSPAAIAEFALTDTLYLLRNMGKVQAQLQAGDYEKAGTFIGKELGQQTVGVMGTGHIGQVAIKLFKGFGAKVIAYDPYPMKGDHPDFDYVSLEDLFKQSDVIDLHVPGIEQNTHIINEAAFNLMKPGAIVINTARPNLIDTQAMLSNLKSGKLAGVGIDTYEYETEDLLNLAKHGSFKDPLWDELLGMPNVVLSPHIAYYTETAVHNMVYFSLQHLVDFLTKGETSTEVTGPAK

SEQ ID NO: 8 - protein, 333 aa, Alcaligenes faecalis
MKPRIVTTHRIHPDTLALLETAAEVISNQSDSTMSREEVLLRTNDADGMMVFMPDSIDADFLSACPNLKVIGAALKGYDNFDVEACTRHGIWFTIVPDLLTSPTAELTIGLLLSITRNMLQGDNYIRSRQFNGWTPRFYGTGLTGKTAGIIGTGAVGRAVAKRLAAFDMQIQYTDPQPLPQESERAWNASRTSLDQLLATSDFIIPMLPMSSDTHHTINARALDRMKPGAYLVNACRGSIVDERAVVHALRTGHLGGYAADVFEMEEWARPDRPHSIPDELLDPALPTFFTPHLGSAVKSVRMEIEREAALSILEALQGRIPRGAVNHVGAGR

SEQ ID NO: 9 - DNA, 1006 bp, Alcaligenes faecalis
atgaaaccaagaatcgtaacaacgcatcgaatccaccctgacacgctggccctccttgagaccgccgctgaagtaatttccaatcagtccgattcaaccatgtcgcgggaagaggtactgttgcgcaccaatgatgcggacgggatgatggtgtttatgccggatagcatagatgcggattttctatccgcgtgccccaatctgaaggtcatcggcgccgcgcttaaaggatatgacaactttgatgtcgaggcatgtacccgccatgggatttggtttacgattgttcctgatttgcttacgagtcccacagctgaactaacgattggcctgttgctgagcatcacacggaatatgctgcaaggtgataattacattcgatcacgccagttcaatggttggaccccgcggttttatggcacaggtttgacgggtaaaaccgccggcatcattgggacgggagcggtcgggcgggcggtcgcaaagcggctggccgccttcgatatgcaaattcagtacacggatccgcagcctttgccgcaagagtcggaaagggcatggaatgcgagcagaacatcgctggaccagctattggcgacaagcgatttcatcattcccatgctgccgatgtcgtcagatacccaccacaccataaatgctcgggcattagaccgcatgaagcccggtgcgtacctcgtcaatgcctgtcgcggctccatcgtagatgagcgggccgtggtgcatgcgcttcggacggggcatctgggcggttacgccgcagacgtcttcgagatggaggaatgggcgcgtcccgacaggccgcattctattcctgacgaattgcttgatcctgctttacccacattctttacgcctcacctgggttcggcggtcaaatcggtacggatggaaatcgagcgtgaagccgccctcagtatcctcgaagcgttgcaagggcgcattccacgcggagcggtcaaccatgttggagcggggagatgactga

SEQ ID NO: 10 - protein, 331 aa, Xanthobacter flavus
MARKTIVTNWVHPEVLDLLSTRGPAEANTTREPWPRDEIIRRAHGADAMLAFMTDHVDAAFLDACPELKIVACALKGADNFDMEACRARKVAVTIVPDLLTAPTAELAVGLMITLGRNLLAGDRLIRERPFAGWRPVLYGTGLDGAEVGIVGMGAVGQAIAHRLRPFRCRLSYCDARPLSPAAEDAQGLLRRDLADLVARSDYLVLALPLTPASRHLIDAAALAGMKPGALLINPARGSLVDEAAVADALEAGHLGGYAADVFETEDWARPDRPAAIEARLLAHPRTVLTPHIGSAVDSVRRDIALAAARDILRHLDGLQQDPPSRDRSAG

SEQ ID NO: 11 - DNA, 996 bp, Xanthobacter flavus
atggcccgcaagaccatcgtcaccaattgggtgcaccccgaggtgctggacctgctttccacccgcggcccggcggaggccaacaccacccgcgaaccgtggccgcgcgacgagatcatccgccgcgcccacggcgcggacgccatgctcgccttcatgaccgaccatgtggacgccgccttcctcgacgcctgcccggagctgaagatcgtcgcctgcgccctgaagggggccgacaatttcgacatggaggcgtgccgtgcccgcaaggtggcggtgaccatcgtgccggacctgctcaccgcgccgacggcggagctggcggtgggcctcatgatcaccctcggccgcaacctgctcgccggcgaccggctgatccgcgaacggccgttcgccggctggcggccggtgttgtacggcaccggcctcgacggggcggaggtgggcatcgtcggcatgggcgcggtggggcaggccatcgcccaccgcctgcggccgttccgctgccgcctctcctattgcgacgcgcggcccctctcccccgcagcagaagatgcccaggggctgctgcggcgcgacctcgccgacctcgtcgcgcgcagcgactacctggtgctggccctgccgctgacacccgcctcgcgccacctgatcgacgccgcggcccttgcgggcatgaagccgggcgcgctgctcatcaatcccgccaggggctcgctggtggacgaggcggcggtggccgatgccctggaggccggccatctcggcggctatgcggccgatgtgttcgaaaccgaggactgggcccgccccgatcgccccgccgccatcgaggcgcgcctcctcgcccatccgcgcacggtgctcaccccgcacatcggatcggcggtggacagcgtgcggcgggacatcgcgcttgccgcggcgcgggacatcctgcgccatctggacggattgcagcaggacccgccttcgcgcgatcgttcggcggcctga

SEQ ID NO: 12 - protein, 336 aa, Pseudomonas putida
  strain WM1639
MLPKLVITHRVHDEILQLLAPHCELMTNQSDSTLTREEILRRCRDAQAMMAFMPDRVDAEFLQACPELRVVGCALKGFDNFDVDACTARGVWLTFVPDLLTVPTAELAIGLAVGLGRHLRAADAFVRSGEFQGWQPQFYGTGLDNATVGILGMGAIGQAMAERLQGWGATLQYHEAKALDTQTEQRLGLRQVACSELFASSDFILLALPLNADTEHLVNAELLALVRPGALLVNPCRGSVVDEAAVLAALERGQLGGYAADVFEMEDWARADRPRLIDPALLAHPNTLFTPHIGSAVRAVRLEIERCAAQSIIQALAGARPINAANRLPQAEPAAC

SEQ ID NO: 13 - protein, 336 aa, Klebsiella ornithinolytica; identical to SEQ ID NO: 1
  strain WM1686

SEQ ID NO: 14 - DNA, 1011 bp, Klebsiella ornithinolytica
  strain WM1686
atgctgccgaaactcgttataactcaccgagtacacgatgagatcctgcaactgctggcgccacattgcgagctgatgaccaaccagaccgacagcacgctgacgcgcgaggaaattctgcgccgctgtcgcgatgctcaggcgatgatggcgttcatgcccgatcgggtcgatgcagactttcttcaagcctgccctgagctgcgtgtagtcggctgcgcgctcaagggcttcgacaatttcgatgtggacgcctgtactgcccgcggggtctggctgaccttcgtgcctgatctgttgacggtcccgactgccgagctggcgatcggactggcggtggggctggggcggcatctgcgggcagcagatgcgttcgtccgctctggcgagttccagggctggcaaccacagttctacggcacggggctggataacgctacggtcggcatccttggcatgggcgccatcggactggccatggctgatcgcttgcagggatggggcgcgaccctgcagtaccacgaggcgaaggctctggatacacaaaccgagcaacggctcggcctgcgccaggtggcgtgcagcgaactcttcgccagctcggacttcatcctgctggcgcttcccttgaatgccgatacccagcatctggtcaacgccgagctgcttgccctcgtacggccgggcgctctgcttgtaaacccctgtcgtggttcggtagtggatgaagccgccgtgctcgcggcgcttgagcgaggccagctcggcgggtatgcggcggatgtattcgaaatggaagactgggctcgcgcggaccggccgcggctgatcgatcctgcgctgctcgcgcatccgaatacgctgttcactccgcacatagggtcggcagtgcgcgcggtgcgcctggagattgaacgttgtgcagcgcagaacatcatccaggtattggcaggtgcgcgcccaatcaacgctgcgaaccgtctgcccaaggccgagcctgccgcatgttga

SEQ ID NO: 15 - protein, 336 aa, Klebsiella oxytoca; identical to SEQ ID NO: 1
  strain WM1733

SEQ ID NO: 16 - DNA, 1011 bp, Klebsiella oxytoca; identical to SEQ ID NO: 14
  strain 1733

SEQ ID NO: 17 - protein, 336 aa, Pseudomonas stutzeri; identical to SEQ ID NO: 12
  strain WM2048

SEQ ID NO: 18 - DNA, 1011 bp, Pseudomonas stutzeri
  strain 2048
atgctgccgaaactcgttataactcaccgagtacacgatgagatcctgcaactgctggcgccacattgcgagctgatgaccaaccagagcgacagcacgctgacgcgcgaggaaattctgcgccgctgccgcgatgctcaggcgatgatggcgttcatgcccgatcgggtcgatgcagaatttctccaagcctgccccgagctgcgtgtggtcggctgcgcgctcaagggcttcgacaatttcgatgtggacgcctgtactgcccgcggggtctggttgaccttcgtgcctgatctgttgacggtcccgactgccgagctggcgatcggactggcggtggggctggggcggcatctgcgcgcagcagatgcgttcgtccgctctggcgagttccagggctggcaaccacagttctacggcacggggctggataacgcgacggtcggcatccttggcatgggcgccatcggacaggccatggctgagcgcttgcagggatggggcgcgaccctgcagtaccacgaggcgaaggctctggatacacaaaccgagcaacggctcggcctgcgccaggtggcgtgcagcgaactcttcgccagctcggacttcatcctgctggcgcttcccttgaatgccgatactgagcatctggtcaacgccgagctgcttgccctcgtacggccgggcgctctgcttgtaaacccctgtcgtggttcggtagtggatgaagccgccgtgctcgcggcgcttgagcgaggccagctcggcgggtatgcggcggatgtattcgaaatggaagactgggctcgcgcggaccggccgcggctgatcgatcctgcgctgctcgcgcatccgaatacgctcttcactccgcacatagggtcggcagtgcgcgcggtgcgcctggagattgaacgttgtgcagcgcagagcatcatccaggcgttggcaggtgcgcgcccaatcaacgctgcgaaccgtctgccccaggccgagcctgccgcatgttga

SEQ ID NO: 19 - protein, 330 aa, Nostoc punctiformae
MKPKVVITHWVHPEIITNLSEYCEVVANPTRETLPREEILKLAQDAEALMVFMPDRIDEAFLKACPKLKIIAGALKGYDNFDVDACTRQGIWFTIVPSLLAVPTAELTIGLIIGLARQMLLGDRLIRQGTFAGWRPHLYGMGLANRTLGIVGMGSLGQALAQRLSSFEMNLIYTDAIPLPKEKAAAWCLSQVSLDTLLATSDFVVLMVPLQPETFHLINEKSLARMKPGSFLINPCRGSVVDEQAVSDALASGHLAGYAADVFELEDWARSDRPSKIPPSLLEKQDQTFFTPHLGSAVDDLRYDIAIEASQNILQVLQGHSPQGAINCPS

SEQ ID NO: 20 - protein, 332 aa, Nostoc sp.
  strain PCC1720
MKPKVVITNWVHPEVIELLKPSCEVIANPSKEALSREEILQRAKDAEALMVFMPDTIDEAFLRECPKLKIIAAALKGYDNFDVAACTHRGIWFTIVPSLLSAPTAEITIGLLIGLGRQMLEGDRFIRTGKFTGWRPQFYSLGLANRTLGIVGMGALGKAIAGRLAGFEMQLLYSDPVALPPEQEATGNISRVPFETLIESSDFVVLVVPLQPATLHLINANTLAKMKPGSFLINPCRGSVVDEQAVCKALESGHLAGYAADVFEMEDWYRSDRPHNIPQPLLENTKQTFFTPHIGSAVDELRHNIALEAAQNILQALQGQKPQGAVNYLRES

SEQ ID NO: 21 - protein, 331 aa, Tricodesmium sp.
MDKKPLVVITHWIHSQVIDYLSKTCEVILNQTQYTLSREEIINRTKEAEGLMVFMPDFIESSFLDACPQLKVIAGALRGYDNFDIDACTKRGIWFTIVPDLLAAPTAELTVGLLLGLARRMLEGDRLIRDGQFQGWKPKLYSRGLLHQNLGIVGMGKLGKALAQRLVGFNMNLFYSDSIPLSVEQETAWKISRVSLEELLKISDYVVLMVPLIPDTYHLINQDTLAKMKPKSFLINACRGSVVDEKAIGSAIQSGHLAGYAADVFEMEDRAIPHRPQGIYNTLITEKNKTFLTPHLGSAVDEIRHDISFEAAKNIVQVLSGEIPQGAVNKL

SEQ ID NO: 22 - protein, 327 aa, Ralstonia sp.
MNHRKIVVTQPVHEEVLRKLQAEGEVIMNPGPDPWSPSQLREYLVDADAMMAFMTDSVTKESLLNAPRLKTISCALKGYDNFDLRACAQAGVSVTFVPDLLTEPTAELAIGLAIAAGRNVLQGDAATRAGYSGWRPALYGTGLHGSVASVIGLGKVGQAILARLAGFGCARLLGVDPSVRLDQVELVTLDEAVSTSDYVFLAVPLVSDTRHLVDSRMLQLSKKGQILVNVGRGSVVDERAVVDALANEQLGAYAADVYEMEDWLLPDRPREIHPGLTNNARTVLTPHIGSAVRRVRFEIEMRAAENLVRSLRGESLSDVAVEASAAA

SEQ ID NO: 23 - protein, 336 aa, Klebsiella sp.
MLPKLVITHRVHDEILQLLAPHCELVTNQTDSTLTREEILRRCRDAQAMMAFMPDRVDADFLQACPELRVVGCALKGFDNFDVDACTARGVWLTFVPDLLTVPTAELAIGLAVGLGRHLRAADAFVRSGEFQGWQPQFYGTGLDNATVGILGMGAIGLAMAERLQGWGATLQYHEAKALDTQTEQRLGLRQVACSELFASSDFILLALPLNADTQHLVNAELLALVRPGALLVNPCRGSVVDEAAVLAALERGQLGGYAADVFEMEDWARADRPRLIDPALLAHPNTLFTPHIGSAVRAVRLEIERCAAQNIIQVLAGARPINAANRLPKAEPAAC

SEQ ID NO: 24 - protein, 332 aa, Nostoc sp.; identical to SEQ ID NO: 20
  strain PCC1720

SEQ ID NO: 25 - protein, 330 aa, Nostoc punctiformae; identical to SEQ ID NO: 19

SEQ ID NO: 26 - protein, 331 aa, Tricodesmium erythraeum; identical to SEQ ID NO: 21

SEQ ID NO: 27 - protein, 334 aa, Alcaligenes faecalis
MKPRIVTTHRIHPDTLALLETAAEVISNQSDSTMSREEVLLRTNDADGMMVFMPDSIDADFLSACPNLKVIGAALKGYDNFDVEACTRHGIWFTIVPDLLTSPTAELTIGLLLSITRNMLQGDNYIRSRQFNGWTPRFYGTGLTGKTAGIIGTGAVGRAVAKRLAAFDMQIQYTDPQPLPQESERAWNASRTSLDQLLATSDFIIPMLPMSSDTHHTINARALDRMKPGAYLVNACRGSIVDERAVVHALRTGHLGGYAADVFEMEEWARPDRPHSIPDELLDPALPTFFTPHLGSAVKSVRMEIEREAALSILEALQGRIPRGAVNHVGAGRL

SEQ ID NO: 28 - protein, 336 aa, Klebsiella ornithinolytica; identical to SEQ ID NO: 1
  strain WM1686

SEQ ID NO: 29 - protein, 336 aa, Klebsiella oxytoca; identical to SEQ ID NO: 1
  strain WM1733

SEQ ID NO: 30 - protein, 336 aa, Pseudomonas stutzeri; identical to SEQ ID NO: 1
  strain 2048

SEQ ID NO: 31 - protein, 336 aa, Klebsiella pneumoniae; identical to SEQ ID NO: 23

SEQ ID NO: 32 - protein, 336 aa, Pseudomonas putida; identical to SEQ ID NO: 12
  strain WM1639

SEQ ID NO: 33 - protein, 336 aa, Pseudomonas stutzeri; identical to SEQ ID NO: 12
  strain WM2048

SEQ ID NO: 34 - protein, 331 aa, Xanthobacter flavus; identical to SEQ ID NO: 10

SEQ ID NO: 35 - protein, 327 aa, Ralstonia metallidurans; identical to SEQ ID NO: 22

SEQ ID NO: 36 - protein, 11 aa, Artificial Sequence
  Description of Artificial Sequence Illustrative peptide
GWRPQFYSLGL

SEQ ID NO: 37 - protein, 13 aa, Artificial Sequence
  Description of Artificial Sequence Illustrative NAD binding sequence
GMGALGKAIAGRL

SEQ ID NO: 38 - protein, 13 aa, Pseudomonas stutzeri
GMGAIGLAMADRL

SEQ ID NO: 39 - protein, 13 aa, Artificial Sequence
  Description of Artificial Sequence Formula peptide
GXGXXGXAXXXRL

SEQ ID NO: 40 - protein, 11 aa, Pseudomonas stutzeri
GWQPQFYGTGL

SEQ ID NO: 41 - protein, 11 aa, Artificial Sequence
  Description of Artificial Sequence Formula peptide
GWXPXXYXXGL

SEQ ID NO: 42 - protein, 15 aa, Escherichia coli
MLPKLVITHRVHDEI

SEQ ID NO: 43 - DNA, 28 bp, Artificial Sequence
  Description of Artificial Sequence Primer
ttggatccagagtttgatcmtggctcag

SEQ ID NO: 44 - DNA, 29 bp, Artificial Sequence
  Description of Artificial Sequence Primer
gttggatccacggytaccttgttacgayt

SEQ ID NO: 45 - DNA, 25 bp, Artificial Sequence
  Description of Artificial Sequence Primer
cacacacatatgctgccgaaactcg

SEQ ID NO: 46 - DNA, 20 bp, Artificial Sequence
  Description of Artificial Sequence Primer
agcggataacaatttacagg

SEQ ID NO: 47 - protein, 11 aa, Artificial Sequence
  Description of Artificial Sequence Synthetic peptide
GWRPQFYSLGL

SEQ ID NO: 48 - protein, 11 aa, Artificial Sequence
  Description of Artificial Sequence Synthetic peptide
GWRPHLYGMGL

SEQ ID NO: 49 - protein, 11 aa, Artificial Sequence
  Description of Artificial Sequence Synthetic peptide
GWKPKLYSRGL

SEQ ID NO: 50 - protein, 11 aa, Artificial Sequence
  Description of Artificial Sequence Synthetic peptide
GWTPRFYGTGL

SEQ ID NO: 51 - protein, 11 aa, Artificial Sequence
  Description of Artificial Sequence Synthetic peptide
GWQPQFYGTGL

SEQ ID NO: 52 - protein, 11 aa, Artificial Sequence
  Description of Artificial Sequence Synthetic peptide
GWRPVLYGTGL

SEQ ID NO: 53 - protein, 11 aa, Artificial Sequence
  Description of Artificial Sequence Synthetic peptide
GWRPALYGTGL

SEQ ID NO: 54 - protein, 13 aa, Artificial Sequence
  Description of Artificial Sequence Synthetic peptide
GMGALGKAIAGRL

SEQ ID NO: 55 - protein, 13 aa, Artificial Sequence
  Description of Artificial Sequence Synthetic peptide
GMGSLGQALAQRL

SEQ ID NO: 56 - protein, 13 aa, Artificial Sequence
  Description of Artificial Sequence Synthetic peptide
GMGKLGKALAQRL

SEQ ID NO: 57 - protein, 13 aa, Artificial Sequence
  Description of Artificial Sequence Synthetic peptide
GTGAVGRAVAKRL

SEQ ID NO: 58 - protein, 13 aa, Artificial Sequence
  Description of Artificial Sequence Synthetic peptide
GMGAIGLAMADRL

SEQ ID NO: 59 - protein, 13 aa, Artificial Sequence
  Description of Artificial Sequence Synthetic peptide
GMGAIGLAMAERL

SEQ ID NO: 60 - protein, 13 aa, Artificial Sequence
  Description of Artificial Sequence Synthetic peptide
GMGAIGQAMAERL

SEQ ID NO: 61 - protein, 13 aa, Artificial Sequence
  Description of Artificial Sequence Synthetic peptide
GMGAVGQAIAHRL

SEQ ID NO: 62 - protein, 13 aa, Artificial Sequence
  Description of Artificial Sequence Synthetic peptide
GLGKVGQAILARL